(19) United States
(12) Patent Application Publication
(10) Pub. No.: US 2025/0243446 A1
(43) Pub. Date: Jul. 31, 2025

(54) COLONY COUNTING DEVICE AND CONTROL METHOD

(71) Applicant: Keyence Corporation, Osaka (JP)

(72) Inventor: Shingo MATSUMURA, Osaka (JP)

(73) Assignee: Keyence Corporation, Osaka (JP)

(21) Appl. No.: 18/986,878

(22) Filed: Dec. 19, 2024

(30) Foreign Application Priority Data
Jan. 29, 2024 (JP) 2024-010887

(51) Int. Cl. C12M 1/34; C12M 1/22

(52) U.S. Cl. CPC C12M 41/36; C12M 23/10

(57) ABSTRACT

Burden on a user regarding counting colonies is mitigated. A colony counting device includes an acquisition section that acquires an image of colonies generated in a test individual, and an execution section that executes first software, the execution section executing processing of counting the number of the colonies from the image of the colonies. The execution section reads a user file that holds information in a matrix format is created by second software different from the first software in order to count the colonies generated in the test individual or to manage a count result of the colonies. Moreover, the execution section creates a count table to be used to count the colonies based on the user file.

### CROSS-REFERENCE TO RELATED APPLICATIONS

The present application claims foreign priority based on Japanese Patent Application No. 2024-010887, filed Jan. 29, 2024, the contents of which are incorporated herein by reference.

### BACKGROUND OF THE INVENTION

### 1. Technical Field

The invention relates to a colony counting device, a control method, and a program.

### 2. Description of the Related Art

In a factory that produces food, a colony counter is used to test whether or not bacteria are mixed in a product. An inspector forms a culture medium in a Petri dish, puts a food sample into the culture medium, and cultivates the food sample in a culture vessel or the like for a predetermined period. Thereafter, the inspector takes out the Petri dish from the culture vessel, and counts colonies (bacterial colonies) with the colony counter. In this manner, the counting accuracy of the colony counter is important for food hygiene management.

JP 2015-171334 A proposes counting the number of colonies from a Petri dish image acquired by a camera.

Meanwhile, a user creates a test list and a count table by using widely used spreadsheet software, prints the test list and the count table on paper, and cultures a fungus with a culture medium for each test individual (sample name) while referring to the test list, and counts colonies generated by the fungus visually or using the colony counter. Moreover, the user handwrites a count result of the colonies into the count table. Thereafter, the user opens the original count table with spreadsheet software, and manually inputs the count result from the paper count table to an electronic file. At that time, a mistake in transcription is also likely to occur, and burden of transcription work on the user is heavy.

On the other hand, if a count table is created by a colony counter that can display an electronic count table, the burden of transcription work will be mitigated. In this case, the user has to be familiar with a user interface of the colony counter to create the count table, which is a barrier to introduction of the colony counter. In such a case, if a count table for the colony counter can be created with spreadsheet software familiar to the user, the barrier to introduction of the colony counter will be reduced.

### SUMMARY OF THE INVENTION

Therefore, an object of the invention is to mitigate burden on a user regarding counting of colonies.

The invention provides, for example, a colony counting device including:
an acquisition section configured to acquire an image of colonies generated in a test individual; and
an execution section configured to execute first software, the execution section being configured to execute count processing of a number of the colonies from the image of the colonies,
wherein the execution section includes:
a reading section configured to read a user file that holds information in a matrix format and is created by second software different from the first software in order to count the colonies generated in the test individual or to manage a count result of the colonies; and
a creation section configured to create a count table to be used to count the colonies based on the user file.

According to the invention, the burden on the user regarding the counting of colonies is mitigated.

### BRIEF DESCRIPTION OF THE DRAWINGS

FIG. 1 is a view illustrating a colony counting device;

FIG. 2 is a diagram for describing an electrical configuration of a head device;

FIG. 3 is a diagram for describing an electrical configuration of a control device;

FIG. 4 is a view for describing a user interface (UI);

FIG. 5 is a view for describing a UI for creating a count table from a sample database;

FIG. 6 is a view for describing a UI for newly creating a count table;

FIG. 7 is a view for describing a sheet of spreadsheet software;

FIG. 8 is a view for describing a UI for adding a column element;

FIG. 9 is a view for describing a UI during a test;

FIG. 10 is a view for describing a UI at the time of instructing a count;

FIG. 11 is a view illustrating a UI at the time of registering a count result in a cell;

FIG. 12 is a view for describing a UI illustrating automatic identification of a target cell;

FIG. 13 is a view for describing a UI for re-setting a counting condition;

FIG. 14 is a flowchart illustrating processing executed by a PC;

FIG. 15 is a flowchart illustrating editing of the sample database;

FIG. 16 is a flowchart illustrating a colony counting method;

FIG. 17 is a view for describing a business file;

FIG. 18 is a view for describing a configuration file;

FIG. 19 is a view for describing a function of each cell;

FIG. 20 is a view for describing a count table;

FIG. 21 is a view for describing a count table;

FIG. 22 is a view for describing reflection of a count result;

FIG. 23 is a view for describing handling of a blank cell;

FIG. 24 is a view for describing a Petri dish having a plurality of accommodation regions;

FIG. 25 is a view for describing a configuration file related to the Petri dish having the plurality of accommodation regions;

FIG. 26 is a view for describing referring to a test setting included in the sample DB;

FIG. 27 is a view for describing a tag related to averaging processing;

FIG. 28 is a view for describing a method of converting a configuration file into a count table without using a tag;

FIG. 29 is a view for describing customization of a test setting;

FIG. 30 is a flowchart illustrating a method of creating a count table;

FIG. 31 is a flowchart illustrating a method of acquiring a test column setting; and

FIG. 32 is a flowchart illustrating a method of acquiring a test setting.

### DETAILED DESCRIPTION

Hereinafter, an embodiment will be described in detail with reference to the accompanying drawings. Note that the following embodiment does not limit the invention according to the claims, and all combinations of characteristics described in the embodiment are not necessarily essential for the invention. Two or more characteristics of the plurality of characteristics described in the embodiment may be arbitrarily combined. Further, the same or similar configurations are denoted by the same reference numerals, and redundant description will be omitted.

### First Embodiment

### [Colony Counting Device]

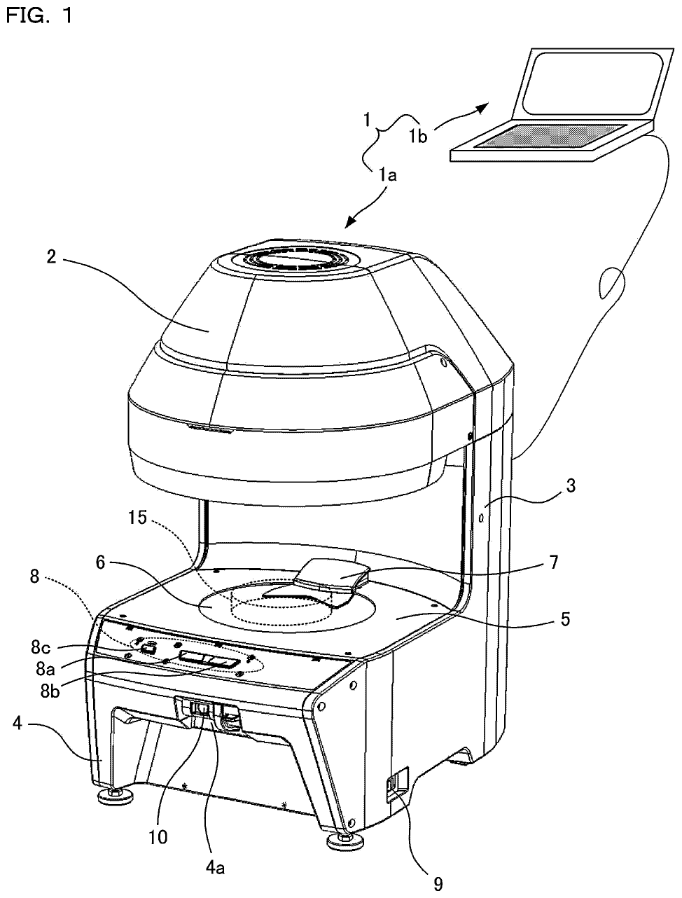

FIG. 1 illustrates a colony counting device 1. Note that the colony counting device 1 includes a head device 1a and a control device (personal computer (PC)) 1b to be described later. For example, the head device 1a and the PC 1b may be connected to each other in a wired manner by a universal serial bus (USB) cable, or may be connected to each other in a wireless manner.

The head device 1a includes an upper unit 2, a support unit 3, and a lower unit 4. A camera and an illumination device are provided inside the head device 1a. The support unit 3 exists between the upper unit 2 and the lower unit 4, and supports the upper unit 2. A stage 5 is provided on a top surface of the lower unit 4. The stage 5 is provided with a transmission window 6 on which a Petri dish 15 is placed and a positioning member 7 configured to position the Petri dish 15 at the center of the transmission window 6. An operation section 8 and a front camera 10 are provided in front of the lower unit 4. The operation section 8 includes a plurality of switches (for example, a first hardware button 8a, a second hardware button 8b, and a third hardware button 8c) configured for a user to input instructions. The front camera 10 is optional, and reads, for example, a two-dimensional symbol (barcode) and the like. The front camera 10 is arranged in a recess 4a provided in a front surface of a housing of the head device 1. A power switch 9 is provided on a side surface of the lower unit 4.

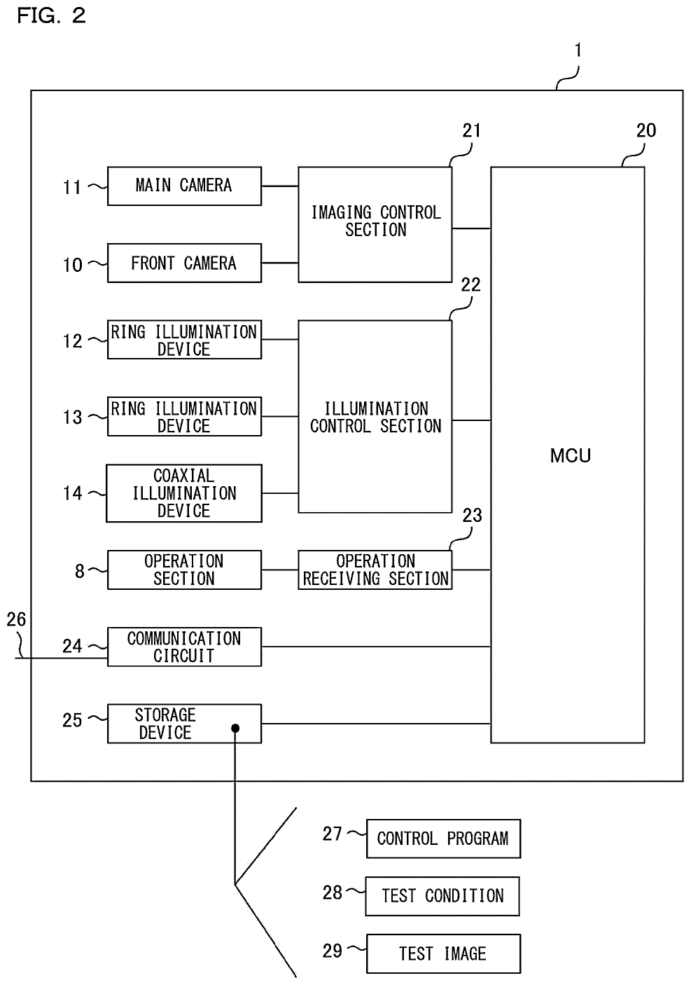

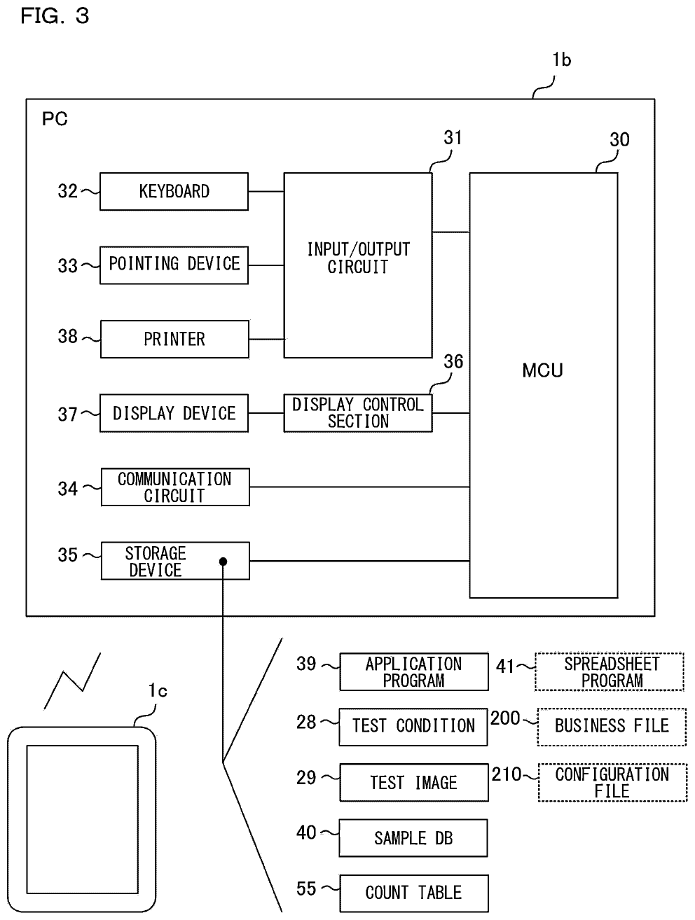

FIG. 2 illustrates an electrical configuration of the head device 1a. An MCU 20 is a processor that executes a control program 27 stored in a storage device 25 and controls the head device 1a according to the control program 27. Note that MCU is an abbreviation for micro-controller unit. The MCU 20 controls the main camera 11 and the front camera 10 via an imaging control section 21 to acquire various types of image data. The imaging control section 21 controls, for example, an exposure time of the main camera 11. The MCU 20 turns on or off the ring illumination devices 12 and 13 and the coaxial illumination device 14 via an illumination control section 22. The illumination control section 22 controls driving power to be supplied to the ring illumination devices 12 and 13 and the coaxial illumination device 14. The MCU 20 receives a user input that is input from the operation section 8 via an operation receiving section 23. The operation receiving section 23 includes an input circuit or the like that generates a signal indicating a state of a switch section of the operation section 8. A communication circuit 24 is a circuit that communicates with the PC 1b illustrated in FIG. 3 via a communication cable 26. The communication circuit 24 may include a wireless communication circuit and a LAN interface circuit. LAN is an abbreviation for local area network. The communication cable 26 may be, for example, a USB cable. The storage device 25 includes, for example, a read-only memory (ROM) that stores the control program 27 and a random access memory (RAM) used as a work area. The storage device 25 may store, for example, a test condition 28 set by the PC 1b, a test image 29 acquired by the main camera 11, and the like. The test condition 28 can include, for example, an illumination condition, an imaging condition, a count condition, and the like. The test image 29 is an image of the Petri dish 15 including a culture medium and a sample.

FIG. 3 illustrates the PC 1b that controls the head device 1a. An MCU 30 is a processor that executes a program stored in a storage device 35 and controls the PC 1b and the head device 1a according to the program. The MCU 30 receives a user instruction from a keyboard 32 and a pointing device 33 connected to an input/output circuit 31. The MCU 30 controls a printer 38 connected to the input/output circuit 31 to print a table or the like on paper. The MCU 30 displays various types of information on a display device 37 via a display control section 36 such as a graphics board. A communication circuit 34 is a circuit that communicates with the head device 1a via the communication cable 26. The communication circuit 34 may include a wireless communication circuit and a LAN interface circuit. The storage device 35 includes, for example, a read-only memory (ROM) that stores a program and a random access memory (RAM) used as a work area. Moreover, the storage device 35 may include a hard disk drive (HDD) and a solid state drive (SSD). The storage device 35 may store an application program 39, the test condition 28, the test image 29, a sample DB 40, a count table 55, and the like. DB is an abbreviation for database. The application program 39 is in charge of, for example, creation and editing of the sample DB 40 and the count table 55, control of the head device 1, and the like. The test condition 28 is set by the MCU 30 according to the application program 39. The test image 29 is received from the head device 1. The sample DB 40 is a database referred to when the count table 55 is created. The count table 55 is a table in which a plurality of cells are arranged in an array, and includes a row element and a column element.

In the PC 1b, the communication circuit 34 may execute wireless communication with a terminal device 1c such as a smartphone or a tablet terminal. The terminal device 1c may display the count table 55 or may display a test list created from the count table 55. The test list includes a Petri dish number, a sample name, a bacterial species, a culture medium, a dilution factor, a culture time, and the like, and is referred to when the user prepares a test individual in the Petri dish 15.

A spreadsheet program 41 is an optional spreadsheet software that has been widely used. A business file 200 and a configuration file 210 created by the spreadsheet program 41 will be described in detail in a second embodiment.

### [Test Procedure]

A general test procedure is as follows.
(1) The user creates a test list by handwriting. The test list includes a plurality of rows, and a Petri dish number, a bacterial species, a dilution factor, a count number, and a comment (a sample product name and the like) can be written in each of the rows. Note that the Petri dish number referred to here is identification information assigned in advance according to a predetermined rule in order to specify a culture condition such as a type of a culture medium or a dilution factor.
(2) The user writes numbers onto lids of Petri dishes according to the test list, or writes numbers written in advance on the Petri dishes into the test list.
(3) The user creates the culture medium according to the dilution factor written in the test list. If the dilution factor is not written in the test list, the user writes an actual dilution factor in the test list.
(4) The user puts (mixes) a sample into the culture medium of each of the Petri dishes. The user writes a name of the sample in a field of the comment of the test list.
(5) The user puts the Petri dishes into a culture vessel.
(6) When a predetermined time has elapsed, the user takes out the Petri dishes from the culture vessel and counts the number of colonies. For example, the user gives a counted mark with an oil-based pen to a position of a colony while looking through the colony from a bottom surface side of the Petri dish. The number of colonies is written in the test list. Note that the user may count the number of colonies for each bacterial species while visually confirming the bacterial species. In this case, the user writes the number of colonies for each bacterial species into the test list for each of the Petri dishes.
(7) The user activates the PC, and reads and inputs numerical values and characters written in the test list to spreadsheet software. The number of colonies is aggregated using a macro function of the spreadsheet software or the like.

In this manner, the test list is created by handwriting in the conventional test procedure, which is extremely troublesome work for the user. Further, if there is an erroneous input when the numerical values or the like written in the test list are transcribed to a sheet of the spreadsheet software, there is a possibility that an aggregation result is also erroneous. Even if the number of colonies can be automatically acquired by a colony counter, there is still a possibility of erroneous writing and erroneous input since all of the creation of the test list, the writing of the number of colonies into the test list, and the transcription from the test list to the sheet of the spreadsheet software are handwritten in the conventional technique.

Therefore, in the present example, it is proposed that an electronic test list is created by the PC 1b, colonies are counted according to an electronic test list, a counting result is directly input to the electronic test list, and input numbers are aggregated. As a result, burden on the user regarding post-processing on colony counting results may be mitigated. Further, the erroneous input may also be reduced, and test accuracy may be improved since handwriting or manual input by the user is reduced.

### [Creation of Test List (Count Table)]

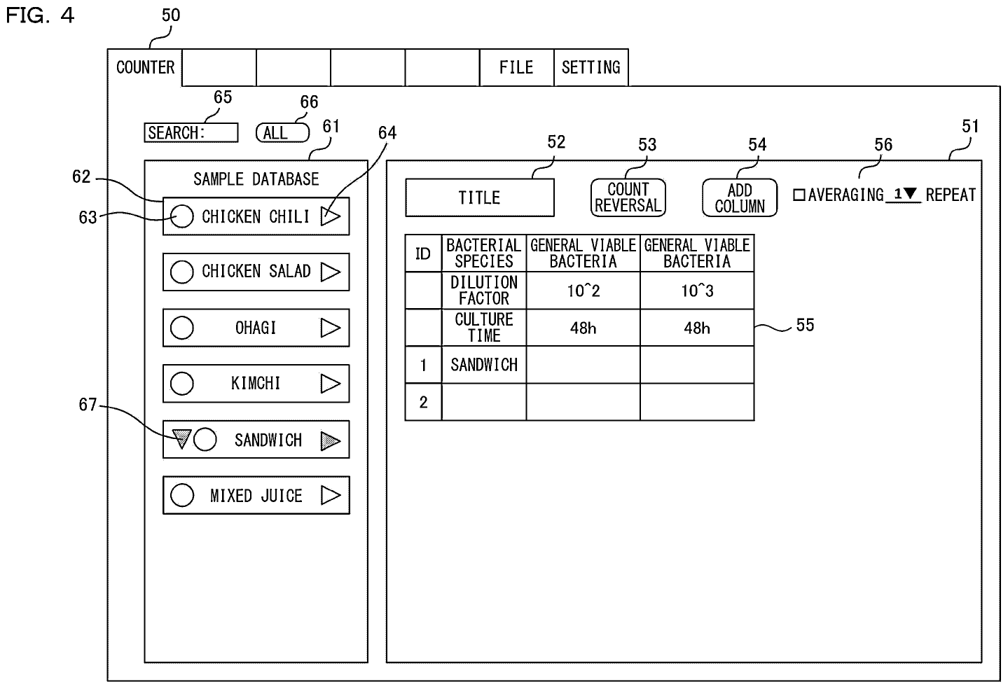

FIG. 4 illustrates an UI 50 of a count application program displayed on the display device 37 of the PC 1b. The count application program is stored in the storage device 35 and executed by the MCU 30. The UI 50 includes a button, a link, a tab, and the like for switching a plurality of functions included in the count application program.

The UI 50 includes a table creation area 51 and a DB display area 61. DB is an abbreviation for database. The table creation area 51 displays at least the count table 55. A title display section 52 receives and displays an input of a title (name) given to the count table 55 from the keyboard 32. A button 53 is a button for switching execution/non-execution of a count of each cell. A button 54 is a button for instructing addition of a column to the count table 55. The averaging setting section 56 includes a check box for instructing whether or not to execute averaging of count results, and a selection section of the number of count values to be averaged (=the number of iterations of the count).

The DB display area 61 displays a list of templates (for example, the sample DB 40) of count items registered in advance. Here, the count item corresponds to one row in the count table 55. The count item is typically distinguished by a name (sample name) of a test target object. A name display section 62 displays a name (sample name) of a template registered in advance. An indicator 63 is an object that visually displays a classification tag associated with the sample name. The classification tag is a tag indicating a classification (for example, a staple, a side dish, or a dessert) defined by a user. For example, the indicator 63 may represent a difference in the classification tag using a difference in a color. The indicator 63 may represent a difference in the classification tag using a difference in a shape of the indicator 63. A button 67 is a button for expanding and displaying one or more sub-items having a parent-child relationship with respect to a certain sample name. The parent-child relationship refers to a relationship between a sample and a plurality of ingredients constituting the sample. For example, when a sandwich is used as a parent, ingredients (for example, ham, lettuce, and egg) constituting the sandwich are children. A button 64 is a button for instructing addition of a corresponding template to the count table 55. Since the sample DB 40 is prepared in advance in this manner, the user can easily create the count table 55.

In a case illustrated in FIG. 4, when the user presses the button 64 associated with a sandwich, the MCU 30 adds a row to the count table 55, displays “1” as an ID in an ID display cell of the added row, and displays “Sandwich” in a cell displaying a sample name in the added row. The ID is an abbreviation for identification information. That is, the ID and the sample name are set on a row-basis, and are “settings for the row”. Moreover, the MCU 30 reads a test condition of the sandwich registered in the sample DB 40 from the storage device 35, adds a new column to the count table 55, and displays the read test condition in the new column. In this example, the test condition includes a bacterial species (for example, general viable bacteria or Escherichia coli), a dilution factor of a culture medium, a culture time of a sample, and the like. Each column includes, for example, a cell for a bacterial species, a cell for a dilution factor, a cell for a culture time, and a cell for a count value. That is, the bacterial species, the dilution factor, and the culture time are set on a column-basis, and are “settings for the column”. In this example, nothing is input to the cell for the count value since a test has not been executed yet and the count value has not been obtained. The first test item for the sandwich is that a culture medium having a dilution factor of 100 times is used for general viable bacteria and a culture time of 48 hours is applied. The second test item for the sandwich is that a culture medium having a dilution factor of 1000 times is used for general viable bacteria and a culture time of 48 hours is applied. In this manner, the MCU 30 adds columns in accordance with the number of the test items.

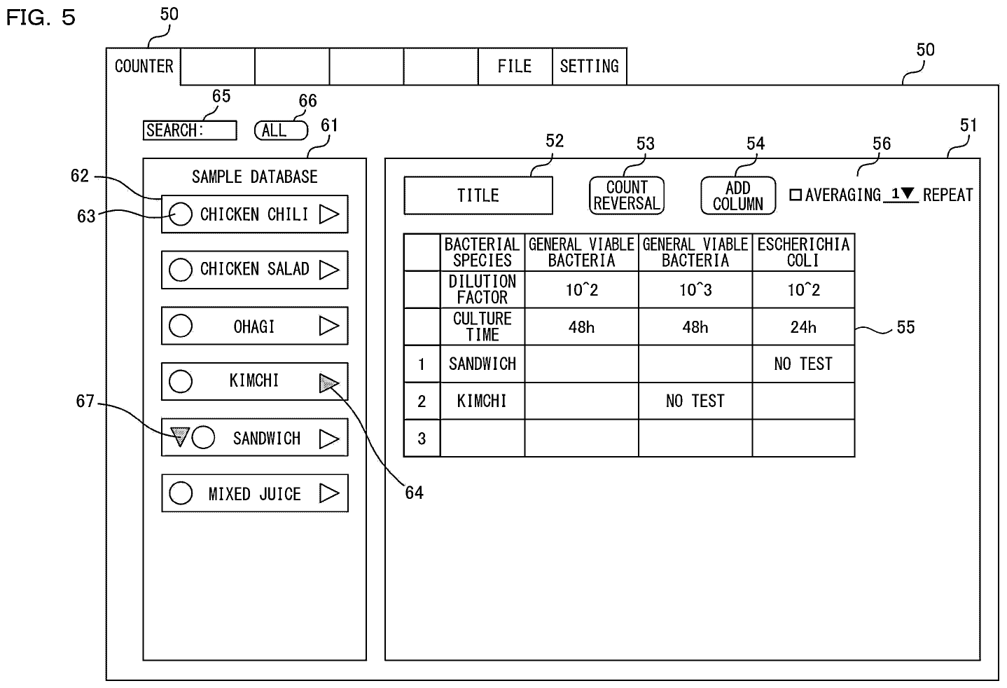

In FIG. 5, when the user presses the button 64 associated with Kimchi, the MCU 30 reads a test condition of the Kimchi registered in the sample DB 40 from the storage device 35, and adds a row and a column corresponding to the read test condition to the count table 55.

In this example, the Kimchi has two test items. The first test item for the Kimchi is that a culture medium having a dilution factor of 100 times is used for general viable bacteria and a culture time of 48 hours is applied. This is common to the first test item for the sandwich. Therefore, the MCU 30 discards the first test item included in a template of the Kimchi and does not add the test item as a new column. The second test item for the Kimchi is that a culture medium having a dilution factor of 100 times is used for Escherichia coli, and a culture time of 24 hours is applied. The MCU 30 adds this as a new column to the count table 55.

Note that a test for Escherichia coli is not performed for the sandwich. Therefore, characters or an image indicating “No test” may be displayed in the cell for the count value. Similarly, a test using the culture medium having the dilution factor of 1000 for general viable bacteria is not performed for the Kimchi. Therefore, the characters or the image indicating “No test” may be displayed in the cell for the count value.

Note that the execution/non-execution of a count can also be executed by operating a count reversal button 53. For the sandwich, when the count reversal button 53 is operated in a state in which the cell corresponding to Escherichia coli is selected, the MCU 30 may be capable of switching between displaying the characters or the image indicating “No test” and leaving a blank to input a count result.

As illustrated in FIGS. 4 and 5, a search box 65 and a tag search narrowing button 66 may be added. When the number of templates registered in the sample DB 40 increases, it becomes difficult for the DB display area 61 to display all the templates at a time. Therefore, the MCU 30 may search the storage device 35 based on characters input to the search box 65 to extract a template, and display a search result in the DB display area 61. Further, when the tag search narrowing button 66 is pressed, the MCU 30 may display only a sample product filtered by a designated classification tag. For example, the same classification tag may be given to a plurality of sample products. In this case, a plurality of sample products given with the selected classification tag are added to the count table 55.

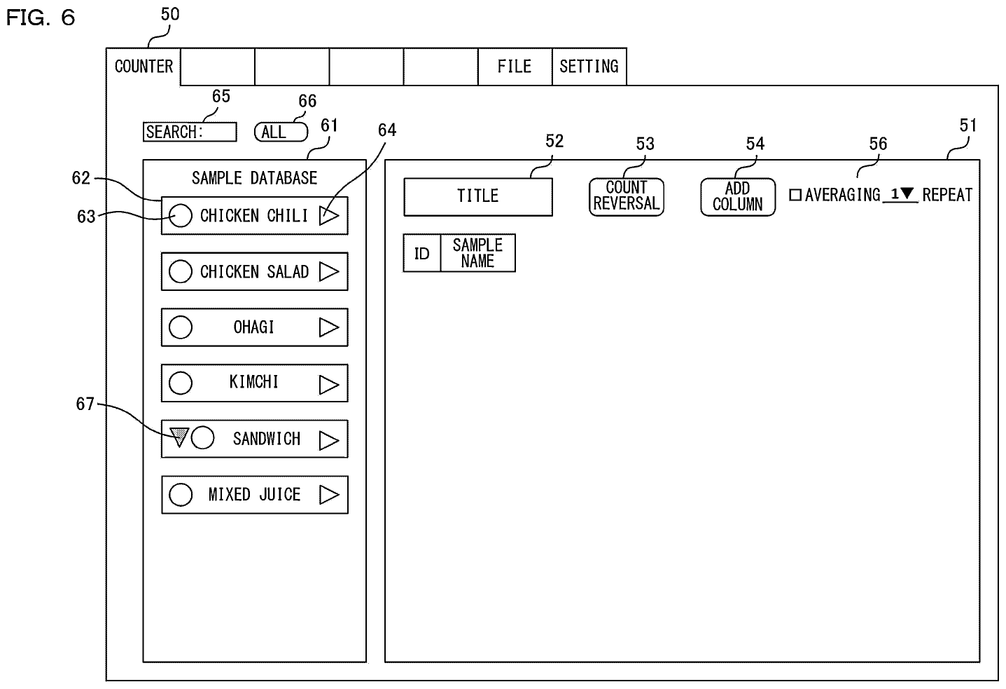

FIG. 6 illustrates the UI 50 in a case where a count table is newly created. For example, the MCU 30 can start spreadsheet software in parallel with the application program 39.

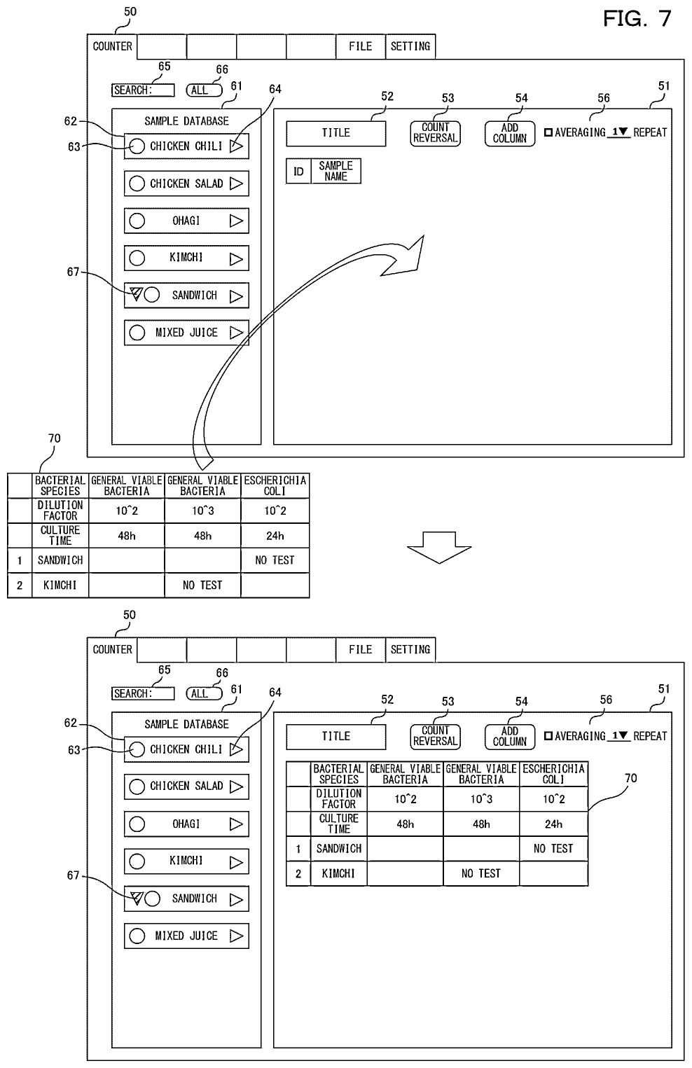

FIG. 7 illustrates a sheet 70 of the spreadsheet software. The MCU 30 receives a copy and paste instruction to the UI 50 for the sheet 70 or a cell group selected in the spreadsheet software. As a result, the MCU 30 may create the count table 55 illustrated in FIG. 5.

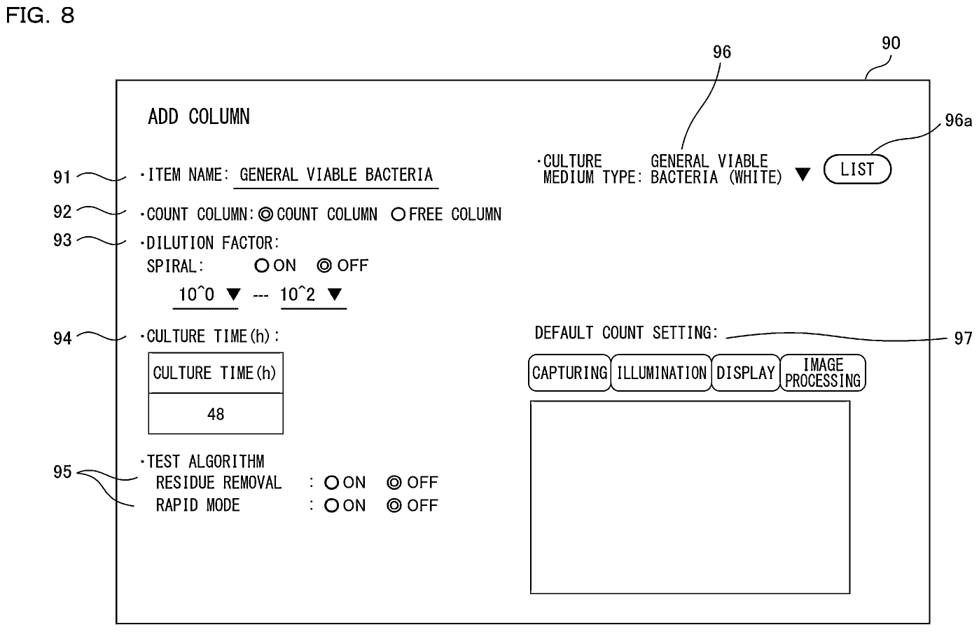

FIG. 8 illustrates a dialog 90 for adding a column. When the button 54 provided on the UI 50 is pressed, the MCU 30 displays the dialog 90 on the display device 37. The item name setting section 91 receives an input of a name of a bacterial species which is an item name of a column. A column type setting section 92 receives a setting as to whether or not a column type is a count column or a free column. The count column is a column including a cell to which a count value is input. The free column is a column in which the user can freely input text, an image, and other information such as a remark and a comment. A dilution factor setting section 93 receives an input of a dilution factor. A culture time setting section 94 receives an input of a culture time. An algorithm setting section 95 receives a setting of image processing to be applied to a test image. Residue removal is a mode in which residues (for example, dirt, stain, and handwriting) attached to the Petri dish 15 or the like are reduced by image processing. A rapid mode is a mode in which rapid result confirmation is emphasized, and is a mode in which the Petri dish 15 cultured in a culture time shorter than that in the related art is tested with higher sensitivity. A culture medium type setting section 96 receives selection of a culture medium type (for example, general viable bacteria (white) or a general viable bacteria (black)) and the like. Note that, when a list button 96a is pressed, the MCU 30 may read culture medium type candidates from the storage device 35 to create a list, and display the list on the display device 37. A count setting section 97 receives a setting of a capturing condition (for example, exposure time) of the main camera 11, a type of illumination (for example, brightness and an illumination device), a type of display processing, a type of image processing, and the like. That is, the bacterial species, the dilution factor, the culture time, the algorithm setting, the culture medium type, or the count setting that has been received in the dialog 90 are set for each column as default settings.

### [Count Processing]

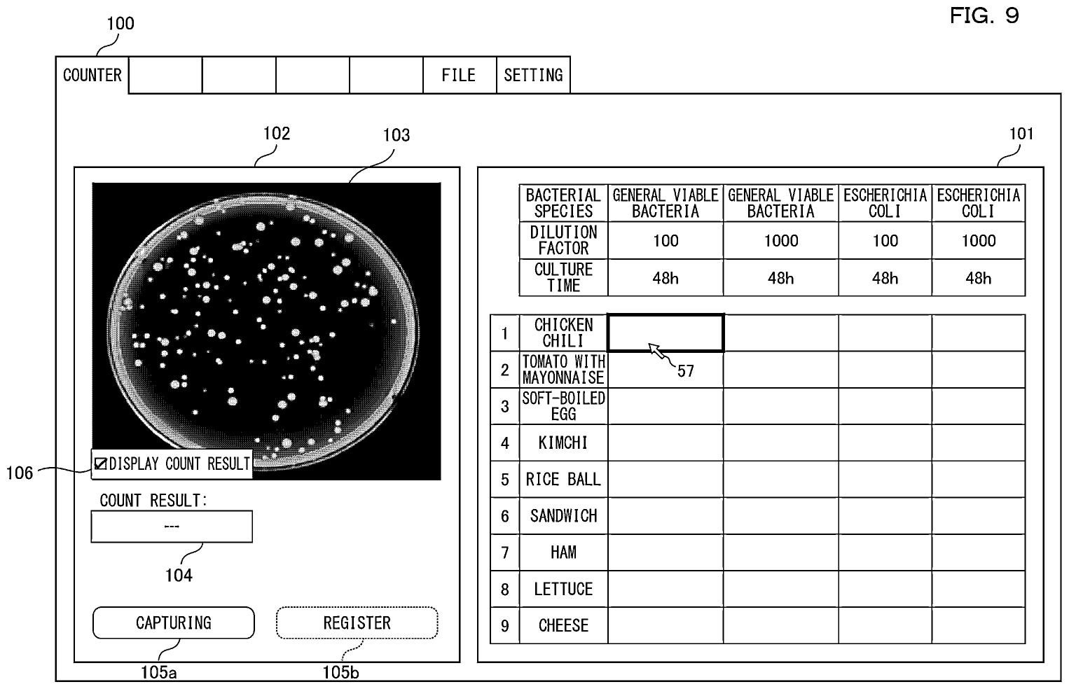

FIG. 9 illustrates a UI 100 displayed on the display device 37 of the PC 1b during execution of count processing by controlling the head device 1a from the PC 1b. A count table area 101 is an area for displaying the count table 55 edited through the UI 50. The result area 102 is an area for displaying a test image 103 acquired by the main camera 11 of the head device 1a. Note that the test image 103 may be a moving image or a still image. In general, the MCU 30 acquires a moving image by the main camera 11 and displays the moving image in the result area 102 when adjustment of the exposure time of the main camera 11, the brightness of each of the ring illumination devices 12 and 13 and the coaxial illumination device 14, selection of light emitting elements to be turned on, selection of the image processing, and the like are executed. On the other hand, the MCU 30 acquires a still image by the main camera 11 and displays the acquired still image in the result area 102 when the count processing is executed.

A check box 106 is a control object for selecting whether or not to display a count result in the count value area 104. A first software button 105a is a button having the same function as the first hardware button 8a. A second software button 105b is a button having the same function as the second hardware button 8b. In this example, a capturing instruction (capture button) is assigned to the first software button 105a. A registration instruction (register button) is assigned to the second software button 105b. In FIG. 9, the second software button 105b is indicated by a broken line, which means being inoperable.

A user clicks and selects a cell corresponding to the Petri dish 15 set on the stage 5 among a plurality of cells included in a count table displayed in the count table area 101 with the pointer 57. As illustrated in FIG. 9, the cell selected by the pointer 57 may be displayed in an emphasized manner such that any cell that has been selected by the user can be recognized. Each cell is stored in the storage device 35 in association with a test condition (sensitivity when binarizing a colony, an illumination device type, brightness, and the like) in advance. The MCU 30 reads the test condition associated with the selected cell from the storage device 35 and transmits the test condition to the head device 1a. The MCU 20 of the head device 1a controls the main camera 11, the ring illumination devices 12 and 13, and the coaxial illumination device 14 according to the received test condition, acquires an image, and transmits the image to the PC 1b. Note that, when another cell is selected, the MCU 30 reads a test condition associated with the selected cell from the storage device 35 and transmits the test condition to the head device 1a. The MCU 20 of the head device 1a controls the main camera 11, the ring illumination devices 12 and 13, and the coaxial illumination device 14 according to the received test condition, acquires an image, and transmits the image to the PC 1b. In this manner, the user can change the test condition by selecting the cell.

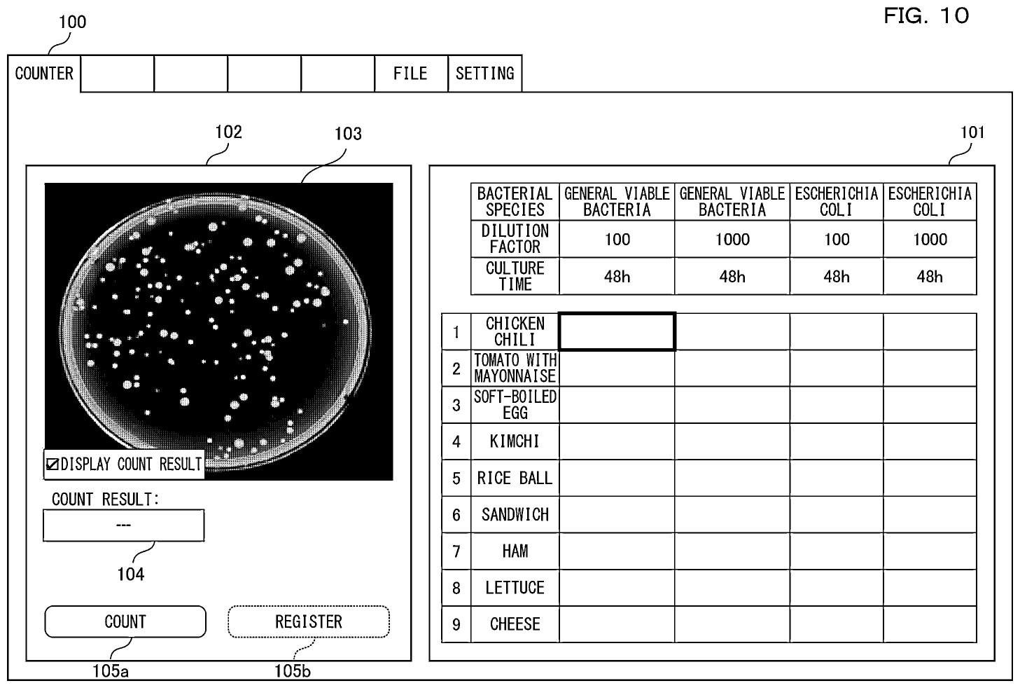

FIG. 10 illustrates the UI 100 displayed on the display device 37 by the MCU 30 when the first software button 105a or the first hardware button 8a, which is the capture button, is pressed. The image 103 (still image) of the Petri dish 15 is displayed in the result area 102. The MCU 30 assigns the first software button 105a from the capture button to a button (count button) for instructing a count.

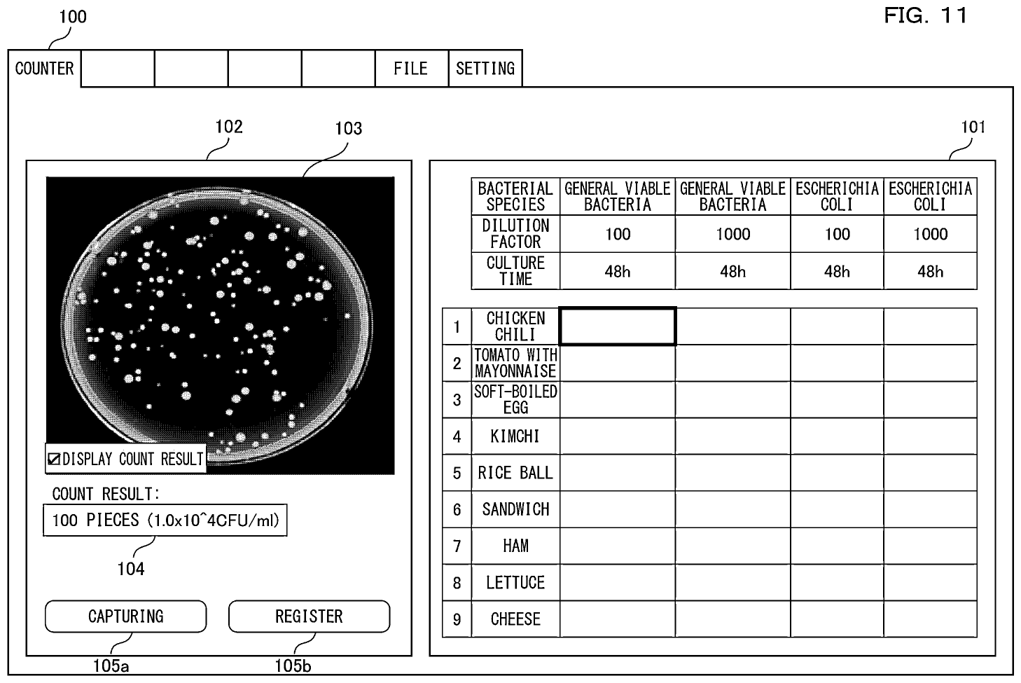

FIG. 11 illustrates the UI 100 displayed on the display device 37 by the MCU 30 when the first software button 105a or the first hardware button 8a, which is the count button, is pressed. When the count button is pressed, the MCU 30 instructs the head device 1a to count colonies. The MCU 20 of the head device 1a counts colonies in response to a count instruction and transmits a count value to the PC 1b. Note that the count processing may be executed by the MCU 30. The MCU 30 displays the count value in the count value area 104. At this time, the MCU 30 may convert the count value into CFU/mL (the number of colonies per section volume (milliliter)) and display CFU/mL in the count value area 104. For example, every time the count value area 104 is clicked with the pointer 57, the MCU 30 may switch display in order of only the count value, only CFU/mL, and the count value+CFU/mL. Note that CFU is an abbreviation for colony forming unit. Further, when the count value is acquired, the MCU 30 re-assigns the first software button 105a from the count button to the capture button. Moreover, the MCU 30 changes the second software button 105b and the second hardware button 8b assigned to the register button from an inoperable state to an operable state.

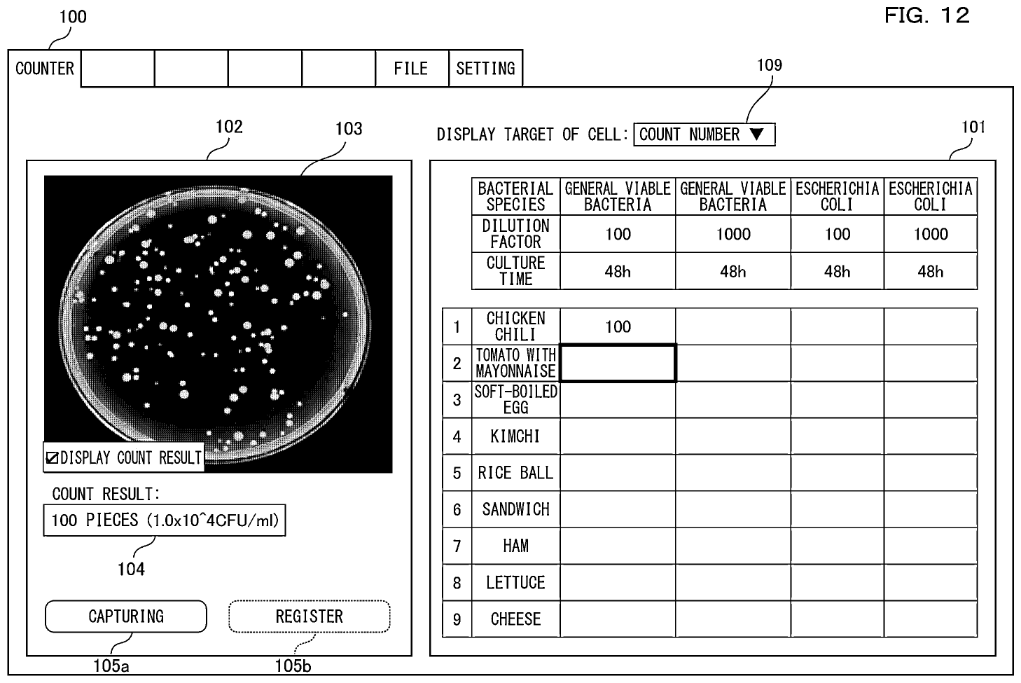

FIG. 12 illustrates a state in which the register button is pressed. The MCU 30 may write a count value to a currently selected cell and change the next cell to a state of being selected (cell of interest). In this example, the cell of interest (active cell) is changed from the cell of the first row to the cell of the second row. In this manner, the MCU 30 automatically selects the next cell, whereby burden on the user is mitigated. Note that the MCU 30 returns the second software button 105b and the second hardware button 8b, assigned to the register button, from the operable state to the inoperable state.

The UI 100 illustrated in FIG. 12 includes a cell display target change menu 109. In FIG. 12, “100” is displayed in a cell since “Count number” is selected in the change menu 109.

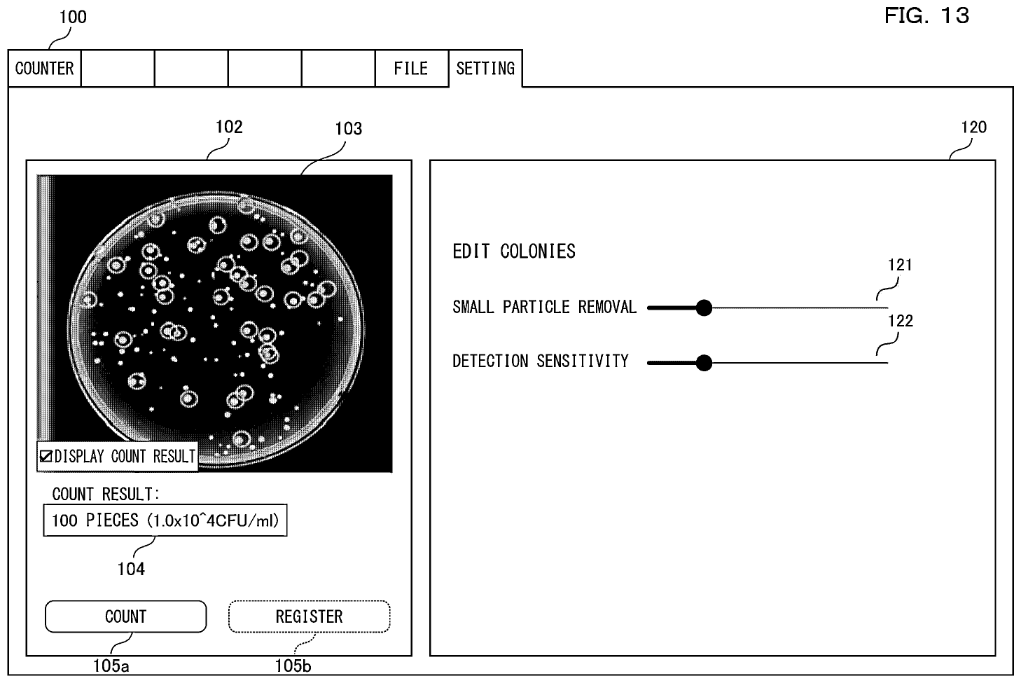

FIG. 13 illustrates the UI 100 displayed on the display device 37 by the MCU 30 when a cell to which a count value has been input is double-clicked. A setting screen 120 includes a control object for adjusting a parameter related to a colony detection algorithm out of a test condition associated with the cell selected by the double click. A slide bar 121 is, for example, a control object for setting a threshold to remove small particles by image processing. A slide bar 122 is a control object for adjusting colony detection sensitivity.

The MCU 30 may display a mark such as a circle to be superimposed on a portion detected as a colony in the image 103 displayed in the result area 102. Since the MCU 30 changes an algorithm according to the adjustment of each of the slide bars 121 and 122, positions and the number of the marks indicating the colonies also change. As a result, the user can easily find an appropriate adjustment amount.

### [Flowchart]

### (1) Main Processing of PC 1b

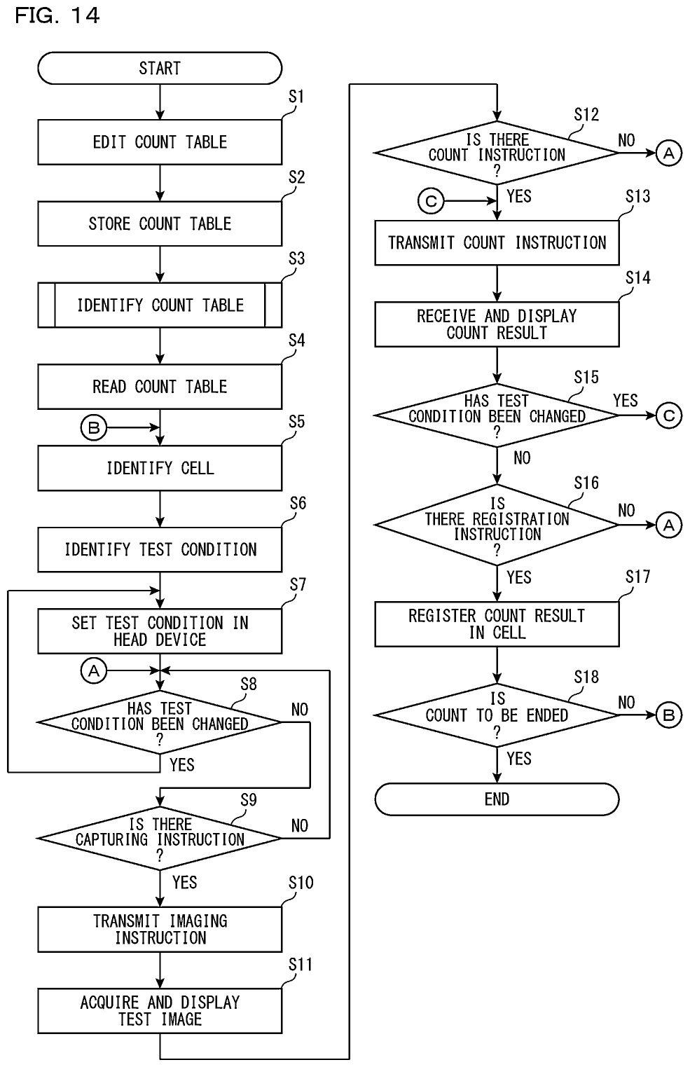

FIG. 14 is a flowchart illustrating a series of processing executed by the MCU 30 of the PC 1b. The MCU 30 executes the following processing according to the count application program stored in the storage device 35.

In S1, the MCU 30 executes editing of a count table. As described with reference to FIGS. 4 to 8 and the like, the count table is edited or created through the UI 50 and the like.

In S2, the MCU 30 stores the count table in the storage device 35.

In S3, the MCU 30 identifies the count table. The count table may be identified using the front camera 10 and the test list or a user authentication tag, or may be identified using a file dialog.

In S4, the MCU 30 reads the identified count table from the storage device 35. As a result, the UI 100 illustrated in FIG. 9 is displayed on the display device 37.

In S5, the MCU 30 identifies a cell to which a count value is to be written. First, a cell in the uppermost row in the count table may be selected, or a cell clicked by the pointer 57 may be selected.

In S6, the MCU 30 identifies a test condition associated with the active cell. For example, the MCU 30 reads the test condition associated with each cell from the storage device 35 when the count table has been created.

In S7, the MCU 30 sets the test condition associated with the active cell in the head device 1a. As described above, the sensitivity of the main camera 11, an illumination device to be turned on, brightness, the number of light emitting elements to be turned on (irradiation direction), image processing (HDR or ring removal), a count algorithm (a parameter such as a threshold), and the like are transmitted to the head device 1a.

In S8, the MCU 30 determines whether or not the test condition has been changed. As described above, the test condition associated with the cell can be changed at any time even during a test. Therefore, when the test condition is changed, the MCU 30 returns to S7 and transmits the changed test condition to the head device 1a. When the test condition is not changed, the MCU 30 proceeds to S9.

In S9, the MCU 30 determines whether or not a capturing instruction has been input by a user. The user can instruct capturing by pressing the first hardware button 8a of the head device 1a or the first software button 105a of the UI 100. When the capturing instruction is not input, the MCU 30 returns from S9 to S8. When the capturing instruction is input, the MCU 30 proceeds from S9 to S10.

In S10, the MCU 30 transmits an imaging instruction to the head device 1a.

In S11, the MCU 30 acquires an image (test image) of the Petri dish image 103 acquired by the main camera 11 from the head device 1a, and displays the test image in the result area 102 of the UI 100.

In S12, the MCU 30 determines whether or not a count instruction has been input. The user can input the count instruction by pressing the first hardware button 8a of the head device 1a or the first software button 105a of the UI 100 assigned as the count button. When the count instruction is not input, the MCU 30 returns from S12 to S8. When the count instruction is input, the MCU 30 proceeds from S12 to S13.

In S13, the MCU 30 transmits the count instruction to the head device 1a. Note that the MCU 30 performs count processing instead of the MCU 20 in a case where the count processing is performed by the PC 1b.

In S14, the MCU 30 receives a count result from the head device 1a, and displays the count result in the count value area 104. Note that, in a case where the MCU 30 executes the count processing in S14, the MCU 30 displays the counting result obtained by executing the count processing in the count value area 104.

In S15, the MCU 30 determines whether or not the test condition such as the image processing and the count algorithm has been changed. When the test condition is changed, the MCU 30 returns to S13. When the test condition is not changed, the MCU 30 proceeds to S16. Note that the change in the test condition in S8 is assumed to be a change in the test condition that requires re-acquisition of an image. The change in the test condition in S15 causes a change in image processing on the acquired image, but it is assumed that re-acquisition of an image is unnecessary.

In S16, the MCU 30 determines whether or not a registration instruction has been input by the user. The user can input the registration instruction by pressing the second hardware button 8b of the head device 1a or the second software button 105b of the UI 100 assigned as the register button. When the registration instruction has not been input, the MCU 30 returns from S16 to S8 to execute re-capturing or change the test condition. When the registration instruction is input, the MCU 30 proceeds from S16 to S17.

In S17, the MCU 30 registers the count result to the active cell.

In S18, the MCU 30 determines whether or not all counts have been ended. For example, when the count results have been input to all the cells existing in the count table, the MCU 30 determines that the counts have been ended. When there is still a cell without any input, the MCU 30 proceeds from S18 to S5, and changes the active cell to the next cell (cell identification).

### (3) Registration of Sample Database

A count table has a plurality of rows and columns, and each cell is associated with a test condition. The count table and a test list may be created again for each day. Meanwhile, there is also a case where a test is executed for the same sample every day. Therefore, burden of count table creation processing is mitigated when a count table is registered in the sample DB 40 in advance for a sample with a high test frequency. Therefore, when a sample table has been created, the user may register a row element corresponding to each sample in the sample DB 40.

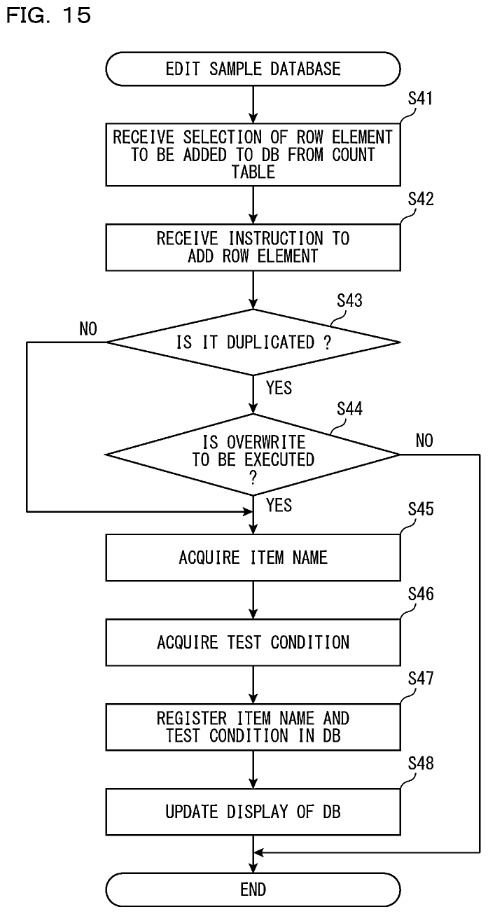

FIG. 15 is a flowchart illustrating editing processing of the sample DB 40 executed by the MCU 30 of the PC 1b. The MCU 30 executes the following processing according to the application program 39 stored in the storage device 35.

In S41, the MCU 30 receives selection of a row element to be registered in the sample DB 40 among a plurality of row elements included in the count table. For example, the MCU 30 may receive a click by the pointer 57 on any row element among the row elements included in the sample table.

In S42, the MCU 30 receives an addition instruction for the selected row element. For example, the addition instruction may be input when a right click is executed by the pointer 57 in a state in which the row element has been selected.

In S43, the MCU 30 acquires a sample name of the row element instructed to be added, and determines whether or not the same sample name has already been registered in the sample DB 40 (duplication determination). When the row element instructed to be added does not already exist, the MCU 30 proceeds from S43 to S45. When the row element instructed to be added exists in the sample DB 40, the MCU 30 proceeds from S43 to S44.

In S44, the MCU 30 inquires of the user whether or not to overwrite the row element in the sample DB 40. When a cancellation instruction is input, the MCU 30 cancels the addition of the row element. When an overwriting instruction is input, the MCU 30 proceeds from S44 to S45.

In S45, the MCU 30 acquires an item name (for example, a sample name, a bacterial species, a culture medium type, or a dilution factor) constituting the row element to be added.

In S46, the MCU 30 acquires a test condition associated with a cell of the row element from the storage device 35.

In S47, the MCU 30 registers the item name and the test condition in the sample DB 40.

In S48, the MCU 30 updates display of the sample DB 40 in the UI 50.

### (6) Count of Colonies

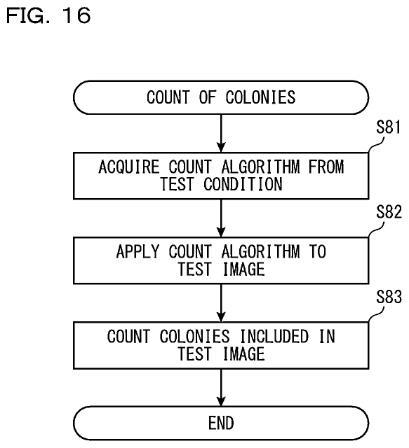

FIG. 16 illustrates colony count processing executed by the MCU 20 of the head device 1a according to the control program. However, image processing and count processing may be executed by the MCU 30.

In S81, the MCU 20 acquires a count algorithm from a test condition received from the PC 1b. Specifically, image processing and a threshold parameter (for example, a binarization threshold) used in the count algorithm are acquired.

In S82, the MCU 20 applies the count algorithm to a test image acquired by the main camera 11. For example, image processing such as HDR or ring removal is applied to the test image.

In S83, the MCU 20 counts colonies included in the test image according to the test condition (threshold parameter).

### Second Embodiment

Meanwhile, among users, there is a user who creates a test list and a count table by using spreadsheet software (the spreadsheet program 41) that has been widely used. There is a case where such a user is required to introduce the colony counting device 1 and transition to an environment in which a test list and a count table are created by the application program 39. In this case, the user sometimes desires to divert the test list and the count table created by the widely used spreadsheet software as assets. Alternatively, it is sometimes desired to create a test list and a count table, or a configuration file serving as a source thereof by using the widely used spreadsheet software in succession.

Therefore, in the second embodiment, a function of converting a user file (configuration file) created by spreadsheet software that has been widely used into a test list and a count table is proposed.

### (1) Procedure

(i) A user creates a test list using the spreadsheet software that has been used. If the test list has already been created, this step is skipped. A format of an electronic file of the test list may be any of CSV format, Excel (registered trademark) format of Microsoft Corporation, and the like.
(ii) The user diverts the test list using the spreadsheet software to create a configuration file on a list to be read by the colony counting device 1. For example, the user adds a cell to the test list using spreadsheet software, and describes tag information in the added cell. The user stores the test list in which the tag information is embedded as the configuration file from the spreadsheet software.
(iii) The PC 1b executes counting of colonies according to the configuration file or by converting the configuration file into a count table for the colony counting device 1.

### (2) User File Created by Spreadsheet Software

### (2-1) Business File (Count Table Also Serving as Test List)

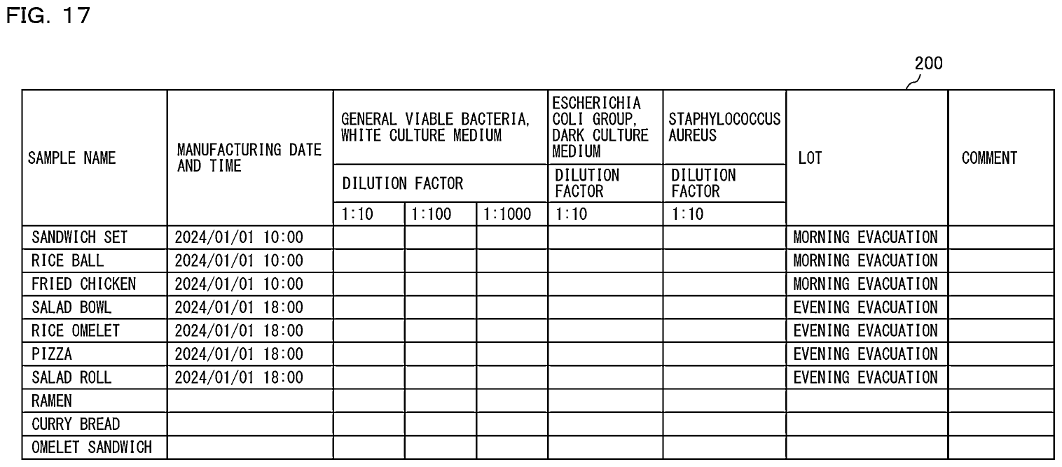

FIG. 17 illustrates the business file 200 created by the user using the spreadsheet program 41. The business file 200 is used as a count table also serving as a test list or simply as a count table. Names of samples (sample names) are described in the leftmost column (column). The manufacturing date and time of each of the samples is described in the second column. The third column describes that a white culture medium is used for general viable bacteria and a dilution factor is 1:10. The fourth column describes that the white culture medium is used for general viable bacteria and the dilution factor is 1:100. The fifth column describes that the white culture medium is used for general viable bacteria and the dilution factor is 1:1000. The sixth column describes that a dark culture medium is used for the Escherichia coli group and the dilution factor is 1:10. The seventh column describes that the dilution factor is 1:10 for Staphylococcus aureus. Lot information indicating a manufacturing lot is described in the eighth column. A comment (remark) is described in the ninth column.

The user generally prints such a business file 200 on paper using a printer and uses the business file 200 as a count table also serving as a test list.

### (2-2) User File (Configuration File) Created Using Spreadsheet Software

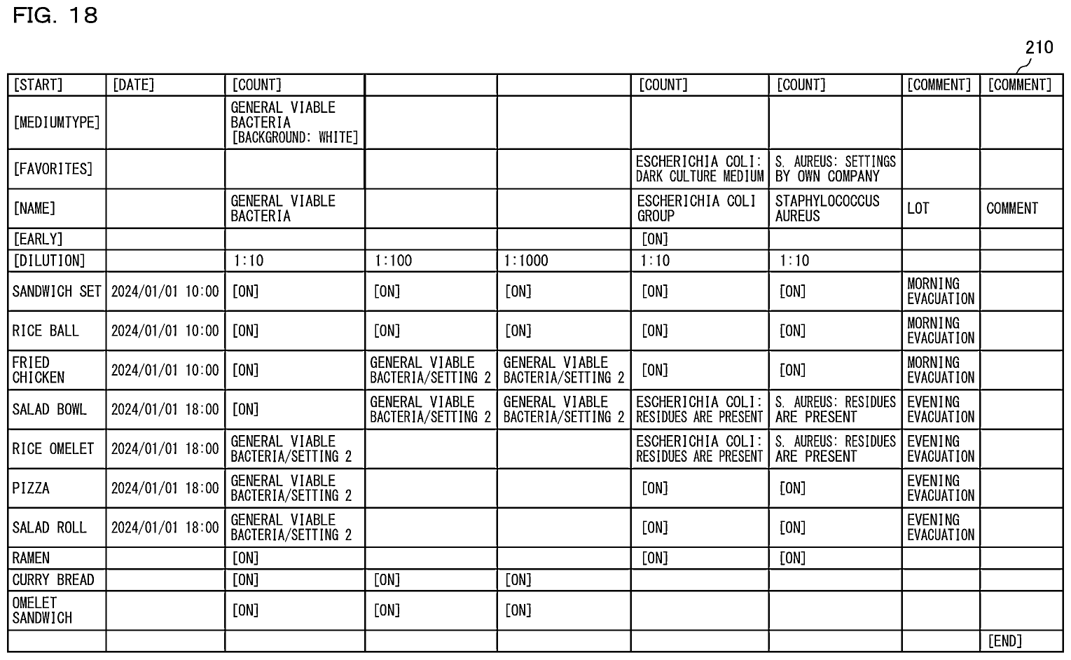

FIG. 18 illustrates the configuration file 210 created by the user using the spreadsheet program 41. The configuration file 210 is created as the user adds a tag and a character string to the business file 200 using the spreadsheet program 41. For example, the user may create the configuration file 210 by copying and renaming the business file 200. In this case, both the business file 200 and the configuration file 210 are maintained in the storage device 35.

A [START] tag is a tag that defines a start position of columns (column elements) and rows (row elements) required to create the count table 55 in the configuration file 210. The start position is expressed as coordinates of a cell, and may be expressed as, for example, (START COLUMN, START ROW). An [END] tag is a tag that defines an end position of the columns and rows required to create the count table 55 in the configuration file 210. The end position is expressed as coordinates of a cell, and may be expressed as, for example, (END COLUMN, END ROW).

A [DATE] tag indicates a column in which date and time information such as the manufacturing date and time is described. A [COUNT] tag indicates a column including a count cell to which a count result of colonies is input. Note that two cells corresponding to the fourth and fifth columns from the left in the first row from the top in FIG. 18 are blank. This is an abbreviated description meaning to follow a tag of a cell on the left side of the corresponding cell. That is, it is indicated that the fourth and fifth columns are also columns each including the count cell according to the [COUNT] tag. A [COMMENT] tag indicates a column to which the user can freely input a character string.

A [MEDIUMTYPE] tag indicates a type of a culture medium and a name of a test setting. Test setting information indicates various parameters (for example, a capturing condition and an image processing condition) set in the colony counting device 1. A [FAVORITES] tag indicates a name of a test setting registered in advance as a favorite by the user among a plurality of test settings. A [NAME] tag indicates a name of a column (such as a name of bacteria) displayed in the count table 55. An [EARLY] tag indicates whether or not a rapid mode is valid. The rapid mode is a mode in which count is executed at a timing when a culture period defined in advance by law ends and at an intermediate timing earlier than the end timing. The rapid mode has an advantage that a sample to be discarded and reproduced can be identified in the middle of the culture period that is long. A [DILUTION] tag indicates the dilution factor. An [ON] tag indicates that a count needs to be executed with a default test setting or indicates the validity of the rapid mode. For example, in FIG. 18, the [ON] tag is described in a row of Sandwich set. This means that a test setting with a name indicated by the [MEDIUMTYPE] tag (“General viable bacteria [background: white]”) needs to be applied to a count cell. It is assumed that “General viable bacteria [background: white]” is associated with General viable bacteria/setting 1. Focusing on the third column, instead of the [ON] tag, “General viable bacteria/setting 2” which is a name of a test setting is input as a character string for Rice omelet. This indicates that “General viable bacteria/setting 2” needs to be applied instead of “General viable bacteria [background: white]” (that is, General viable bacteria/setting 1 which is the default test setting).

Nothing is described in a column with the dilution factor of 1:100 for Rice omelet. This suggests that a count at the dilution factor of 1:100 is not executed for Rice omelet.

As illustrated in FIG. 18, when a colony count is executed for a plurality of culture conditions or bacterial species for one sample name, the [COUNT] tag is described in each of corresponding columns.

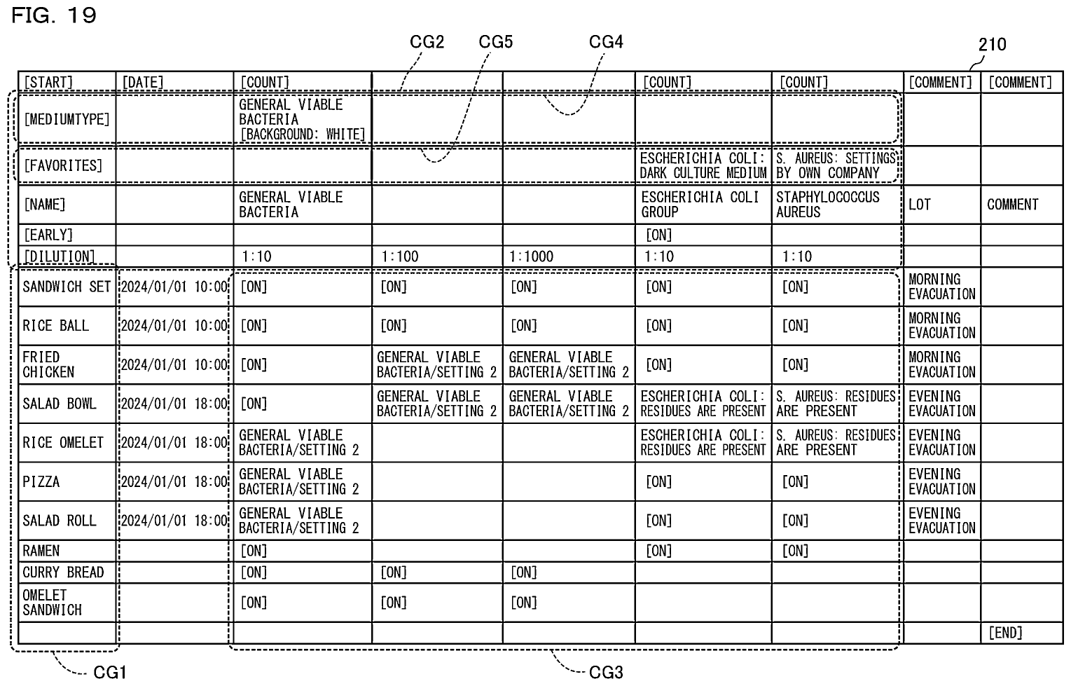

FIG. 19 is a view illustrating cell groups in the configuration file 210.

A cell group CG1 includes one or a plurality of cells in which identification information (sample name) of a test individual is described.

A cell group CG2 includes a cell in which the [DILUTION] tag is described and one or a plurality of cells in which an actual dilution factor is described. The cell group CG2 may also include cell groups CG4 and CG5 since culture conditions are held.

A cell group CG3 includes cells corresponding to the respective count cells in the count table 55. In each of the cells of the cell group CG3, the [ON] tag indicating that a count needs to be executed with a default test setting, any character string indicating that a count needs to be executed with a test setting identified by the described character string, a blank (null character) indicating no execution of a count, or the like is described.

The cell group CG4 includes a cell in which the [MEDIUMTYPE] tag is described and a cell to which a character string indicating a name of the default test setting is input. A blank cell in the cell group CG4 indicates that a test setting of a cell on the right side thereof is valid.

The cell group CG5 includes a cell in which the [FAVORITES] tag is described and a cell to which a character string indicating a name of a favorite test setting is input. A blank cell in the cell group CG5 indicates that the default test setting designated by the [MEDIUMTYPE] tag is valid.

### (3) Count Table

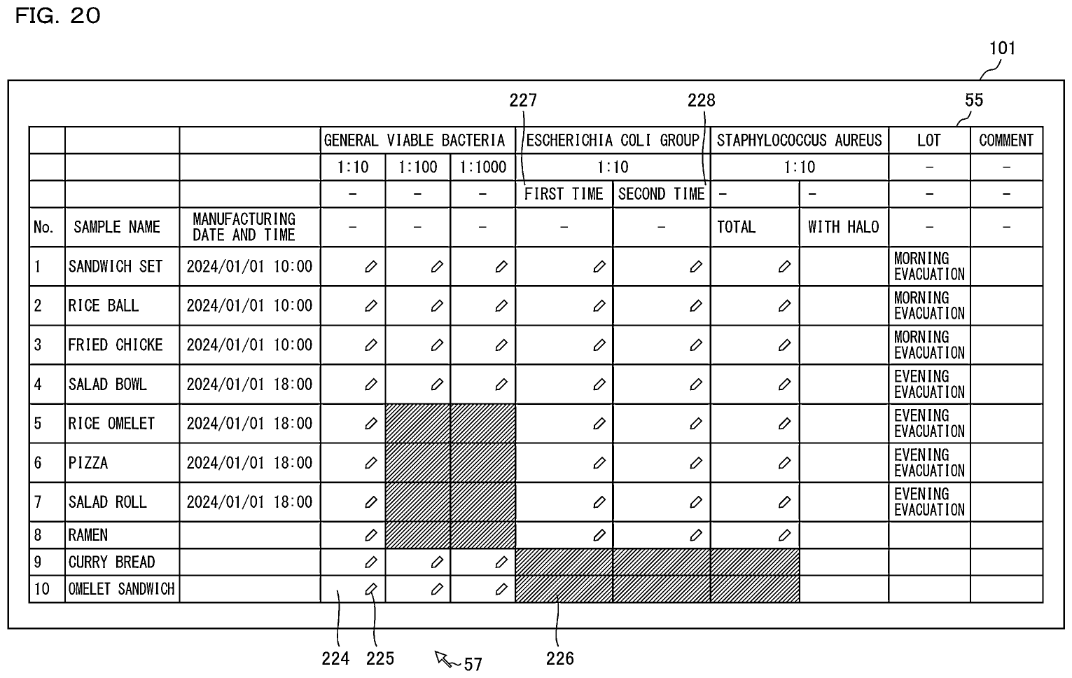

FIG. 20 illustrates that the count table 55 created by converting or analyzing the configuration file 210 is displayed in the count table area 101. When the configuration file 210 illustrated in FIG. 18 is compared with the count table 55 illustrated in FIG. 20, it is understood that a positional relationship between cells of the both is basically maintained. Note that the MCU 30 stores and holds a relationship between a cell in the configuration file 210 and a corresponding cell in the count table 55 in the storage device 35. Further, a test setting is associated with a count cell 224 among the cells in the count table 55. The MCU 30 identifies a test setting based on tag information of a cell in the configuration file 210, and associates the identified test setting with the corresponding count cell 224.

When the configuration file 210 illustrated in FIG. 18 is compared with the count table 55 illustrated in FIG. 20, the MCU 30 copies a sample name described in the cell group CG1 of the configuration file 210 to a column of a sample name in the count table 55. The MCU 30 also copies the manufacturing date and time described in a cell designated by the [DATE] tag in the configuration file 210 to a column of the manufacturing date and time in the count table 55.

The MCU 30 also copies a dilution factor described in the cell group CG2 identified by the [DILUTION] tag as it is to a row of a dilution factor in the count table 55. Further, the MCU 30 also copies a column name designated by the [NAME] tag to a row holding a column name in the count table 55.

Note that the MCU 30 divides a column in which the rapid mode is enabled by the [EARLY] tag and the [ON] tag into two columns. A first column 227 stores a count result of the first time (for example, a time point of 24 h). A second column 228 stores a count result of the second time (for example, a time point of 48 h).

The MCU 30 provides the count cell 224 and a count cell 226 in the count table 55 so as to correspond to the respective cells of the cell group CG3 in the configuration file 210. Here, the MCU 30 may display an icon 225 for confirming or editing the test setting associated with the count cell 224 in the count cell 224 in which a count is to be executed. The icon 225 is clicked by the pointer 57, for example. The MCU 30 grays out the count cell 226 in which a count is not to be executed.

In this manner, in the second embodiment, the business file 200 and the configuration file 210 can be created using the spreadsheet program 41 that is usually used by the user. Moreover, the MCU 30 can convert the configuration file 210 into the count table 55 for the colony counting device 1 according to a conversion or import function installed in the application program 39. As a result, burden for creating the count table 55 is mitigated.

### (4) Another Example of Count Table

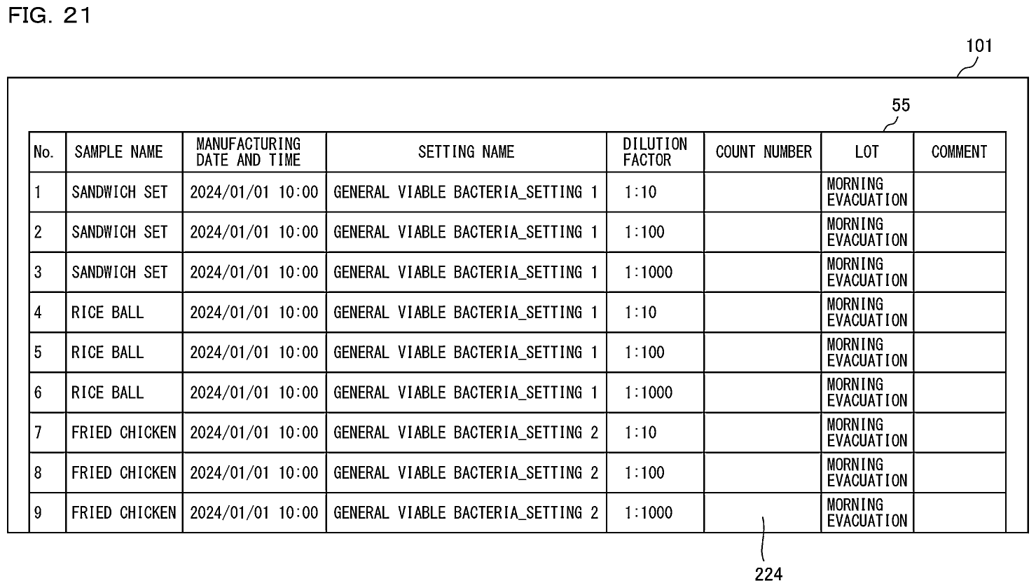

FIG. 21 illustrates another example of the count table 55 created from the configuration file 210. In the count table 55 illustrated in FIG. 20, the plurality of count cells 224 and 226 are arranged in one row for one sample name. However, this is merely an example. As illustrated in FIG. 21, a row to which a count result is input may be created for each combination of a sample name and a dilution factor. In FIG. 21, when there are three combinations of the sample name and the dilution factor, the number of rows to which count results are input is also three. In this manner, an arrangement of cells in the configuration file 210 and an arrangement of cells in the count table 55 do not necessarily coincide with each other.

(5) Reflection of Count Result from Count Table to Configuration File

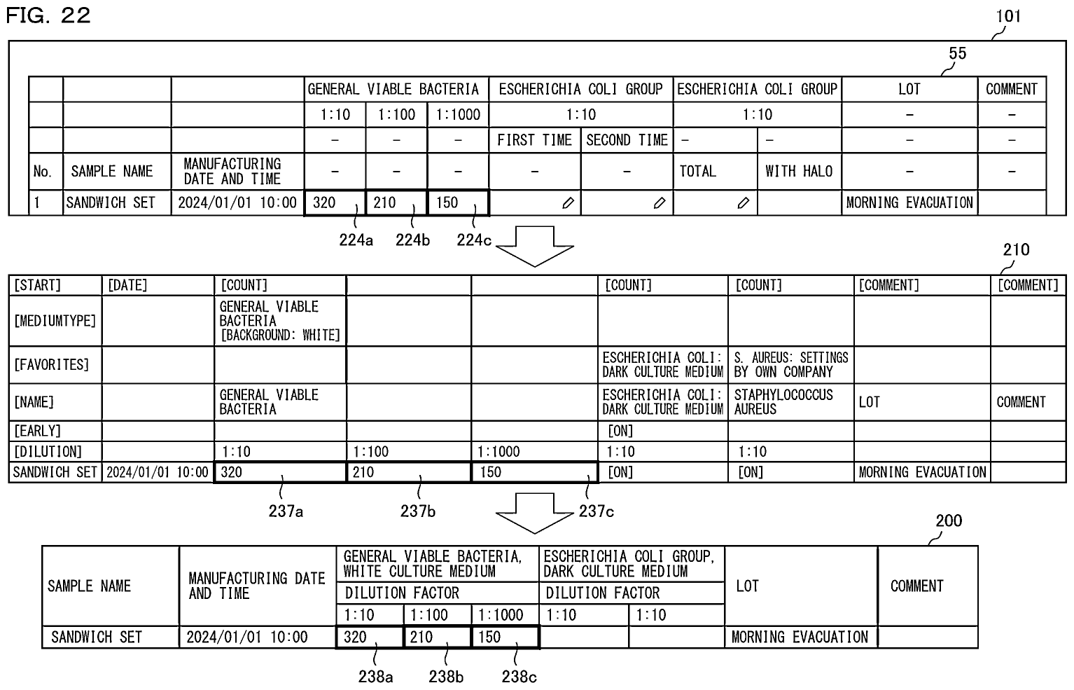

FIG. 22 illustrates a procedure of reflecting count results input to count cells 224a, 224b, and 224c of the count table 55 to the configuration file 210 and the business file 200 by the colony counting device 1. The MCU 30 holds, in the storage device 35, a relationship among the count cells 224a, 224b, and 224c of the count table 55, cells 237a, 237b, and 237c corresponding thereto in the configuration file 210, and cells 238a, 238b, and 238c corresponding thereto in the business file 200. For example, the storage device 35 stores cell-to-cell relationship information indicating that the count cell 224a, the cell 237a, and the cell 238a are related to each other and constitute a cell group. Similarly, the storage device 35 holds cell-to-cell relationship information indicating that the count cell 224b, the cell 237b, and the cell 238b are related to each other and constitute a cell group. Moreover, the storage device 35 also holds cell-to-cell relationship information indicating that the count cell 224c, the cell 237c, and the cell 238c are related to each other and constitute a cell group. The MCU 30 reflects the count results from the count table 55 to the configuration file 210 and the business file 200 while referring to this relationship. That is, the count results input to the count cells 224a, 224b, and 224c are reflected to the cells 237a, 237b, and 237c and the cells 238a, 238b, and 238c, respectively. Note that it has been described that the count result is reflected to the configuration file 210 as an example here. However, this is merely an example. The MCU 30 may output the count result as a new file in a format conforming to a data structure held in an application.

Meanwhile, both the configuration file 210 and the business file 200 are created using the spreadsheet program 41. The spreadsheet program 41 such as Excel (registered trademark) of Microsoft Corporation has a cell reference function. For example, referring to the cell 237a of the configuration file 210 may be described in the cell 238a of the business file 200 (for example, =[configuration file.xlsx] worksheet name! cell coordinates). As a result, the count result copied from the count table 55 to the configuration file 210 may be further input to the business file 200.

### (6) Allowing Blank Row (Blank Cell)

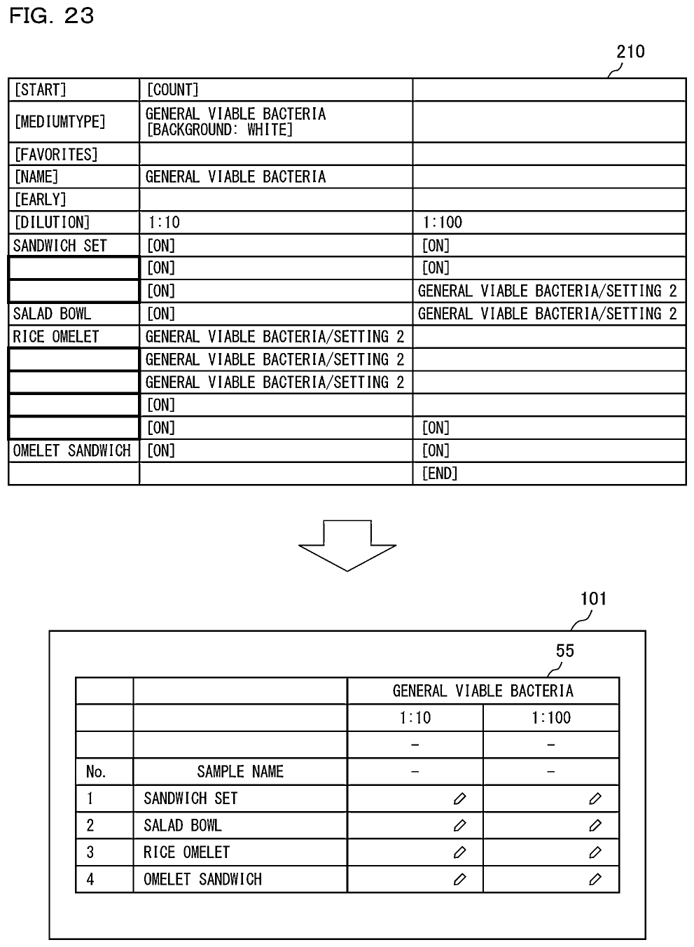

FIG. 23 illustrates processing of converting the configuration file 210 in which a blank cell exists in the cell group CG1 holding the sample name into the count table 55. The user sometimes copies the configuration file 210 created in the past to create a new configuration file 210. Usually, the number of samples as test targets is often constant, but there is a case where some of the samples are missing and are excluded from the test targets. In this case, it would be convenient for the user if the some missing samples can be easily excluded from the test targets.

Therefore, as illustrated in FIG. 23, the user deletes a sample name excluded from the test targets to obtain a blank cell. At this time, the content in test designation cells such as the cell group CG3 is not necessarily deleted. The MCU 30 analyzes the configuration file 210, ignores all the test designation cells existing in the same row (blank row) as a blank cell when the blank cell is found in the cell group CG1, and creates the count table 55.

As illustrated in FIG. 23, a count row (a row including a count cell) corresponding to the same blank row as the blank cell in the cell group CG1 in the configuration file 210 is not provided in the count table 55. As a result, in the count table 55, after a count row of Sandwich set, a count row of Salad bowl is arranged, and a count row of Omelet sandwich is arranged below a count row of Rice omelet.

In this manner, the MCU 30 creates the count table 55 while ignoring the blank cell according to a conversion function of the application program 39. This makes it possible to easily exclude some samples that are missing from the test targets, which is convenient for the user.

Note that character strings of the test designation cells are only ignored even if being left, but this is advantageous when manufacturing of the some samples that have been missing is resumed. This is because it is possible to restore a test setting for a sample name (enable the character strings of the test designation cells) only by rewriting the sample name in the blank cell.

### (7) Division of Petri Dish

The user sometimes uses a Petri dish divided into a plurality of accommodation chambers by a dividing wall. This is convenient for obtaining a plurality of count results having different culture conditions (for example, dilution factors) for the same sample.

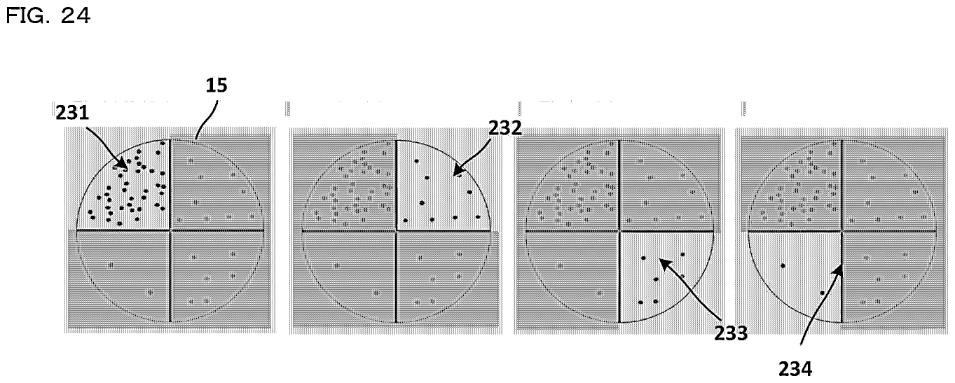

FIG. 24 illustrates the Petri dish 15 divided into M (for example, M=4) effective regions 231 to 234 by dividing walls. In this case, the MCU 30 needs to divide an effective region to be counted in an image of the Petri dish 15 into M effective regions and execute a count for each of the M effective regions. It is complicated and troublesome for the user to do setting work for causing the colony counting device 1 to execute the above.

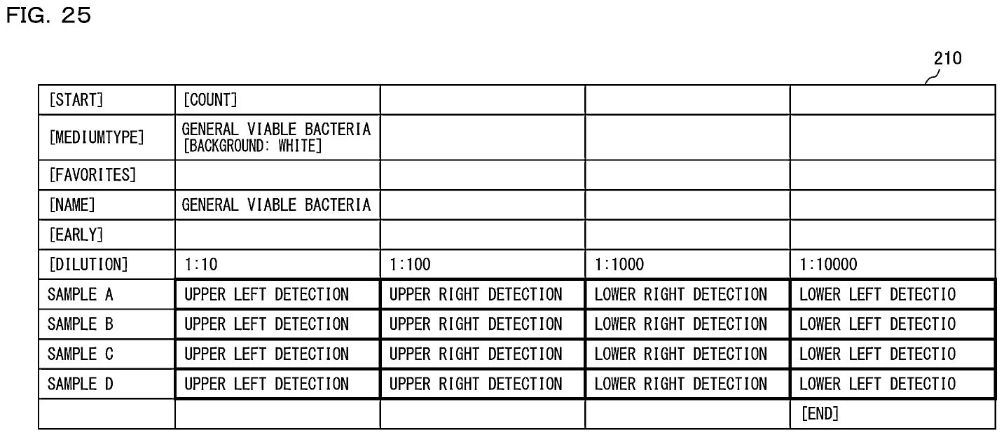

FIG. 25 illustrates a method of designating a customized test setting for each sample. In this example, character strings “Upper left detection”, “Lower left detection”, “Upper right detection”, and “Lower right detection” indicating names of test settings registered in advance as favorites are described in the cell group CG3. The MCU 30 scans the configuration file 210 and associates a name of a test setting with the corresponding count cell 224 in the count table 55 when finding a test designation cell including the character string indicating the name of the test setting in the cell group CG3. When the icon 225 displayed in the count cell 224 is pressed by the user, the MCU 30 reads the test setting from the storage device 35 based on the name of the test setting associated with the count cell 224, applies the test setting to the colony counting device 1, and executes a count. The user can easily create the count table 55 even for a test using the Petri dish 15 divided into the four effective regions 231 to 234 by creating the test settings with the names of “Upper left detection”, “Lower left detection”, “Upper right detection”, and “Lower right detection” in advance.

In this manner, any character string for designating a name of a test setting may be described in a test designation cell in the cell group CG3. That is, the user may directly describe names of test settings in the respective test designation cells without being limited to the Petri dish 15 that has been divided. For example, the user may store a plurality of test settings having different combinations of binarization sensitivity, a threshold for small particle removal, a degree of shape division, a degree of lint removal, sample information (for example, a type of a culture medium, a diameter of a Petri dish, a bacteria name, and an amount of sample solution), and capturing settings (for example, an illumination setting, a brightness setting, enabling/disabling of anti-glare, enabling/disabling of resolution enhancement, and the like) in the storage device 35 in advance, and describe names of these test settings in the test designation cells. As a result, the test setting can be associated with the count cell 224.

### (8) Referring to Sample DB

There is a case where the user desires to constantly associate the same test setting with samples having the same name. If the user remembers a name of a specific test setting as described above, it is sufficient to describe the name in a test designation cell. However, it may be difficult to memorize names of all test settings. Meanwhile, the test settings are associated with sample names in the sample DB 40. Therefore, it would be convenient for the user if the test setting stored in the sample DB 40 for each of the sample names can be associated with the count cell 224 through the configuration file 210.

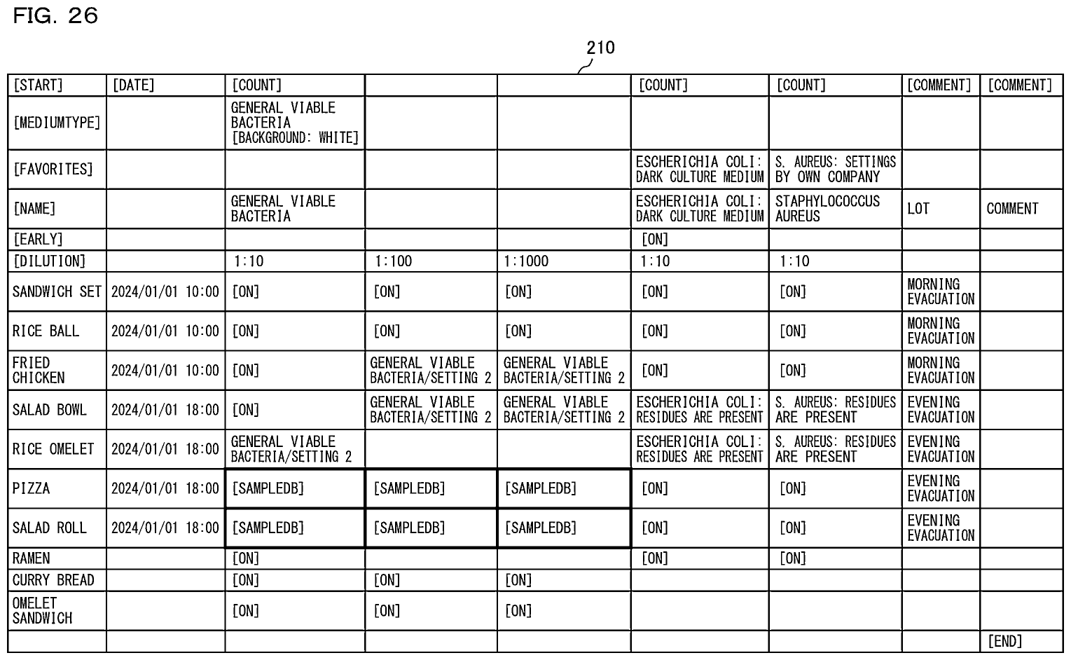

FIG. 26 is a view illustrating a [SAMPLEDB] tag. The [SAMPLEDB] tag is a tag that can be described in a test designation cell. When the [SAMPLEDB] tag is found during scanning of the configuration file 210, the MCU 30 refers to the sample DB 40 based on a sample name existing in a row where the [SAMPLEDB] tag exists, and associates (a name of) a test setting associated with the sample name with the test designation cell in which the [SAMPLEDB] tag is described. More specifically, the MCU 30 associates the test setting held in the sample DB 40 with the count cell 224 of the count table 55 corresponding to the test designation cell. As a result, even if the user does not memorize the name of the specific test setting associated with the sample name, the test setting can be associated with the count cell 224.

### (9) Manufacturing Date and Time and Lot Number

As illustrated in FIG. 18 and the like, the [COMMENT] tag is prepared in the second embodiment. The reason will be described.

There is a case where the user desires to distinguish a plurality of samples having the same sample name by the manufacturing date and time and lot. For example, the user may collectively describe a context (for example, the manufacturing date and time and a lot number) when a series of tests are conducted in a column of “Comment” in the count table 55. In general, it is necessary to input information to the comment column for each Petri dish. Therefore, it is not easy to efficiently input context information managed by the user to the comment column.

Therefore, it is allowed to add column information corresponding to the context to the configuration file 210 in the second embodiment. As a result, addition of data of a type desired by the user is implemented for each sample.

For the manufacturing date and time, the [DATE] tag dedicated to a date and time attribute is prepared. The [DATE] tag means that an input character string is treated as date and time information (date and time attribute). The [DATE] tag enables a search with the manufacturing date and time as a key. Further, when a count result is exported to a spreadsheet file under the control of the spreadsheet program 41, the MCU 30 may assign a time attribute to a column of the manufacturing date and time in the spreadsheet file. This can facilitate data management by the user.

The [COMMENT] tag is a tag for achieving the comment column in the count table 55. In FIG. 18 and the like, a name “Lot” can be assigned to the comment column by further using the [NAME] tag in combination. The MCU 30 may recognize the character string “Lot” as an attribute indicating the lot number or the like.

As illustrated in FIG. 18, by using the [COMMENT] tag and the [DATE] tag, not only the test setting such as the dilution factor but also the lot and the manufacturing date and time of the sample are reflected in the count table 55, and can be managed by the application program 39.

### (10) Averaging Processing

There is a case where it is desired to culture fungi on a plurality of the Petri dishes 15 to which the same test setting (for example, bacterial species and dilution factor) is applied to the same test target, obtain count results in the plurality of Petri dishes 15, and execute averaging processing on the plurality of count results. Therefore, a [REPEAT:n] tag is adopted in the second embodiment. When the [REPEAT:n] tag is found during scanning of the configuration file 210, the MCU 30 recognizes that the same test setting is applied and a count is executed n times, and arranges n count rows to which the same test setting is applied in the count table 55.

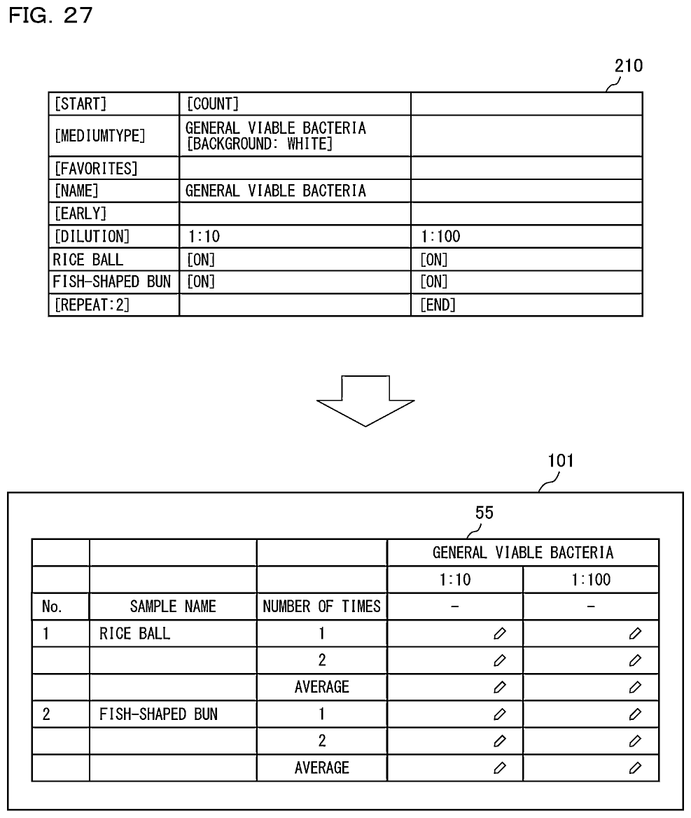

FIG. 27 illustrates a conversion result (the count table 55) of the configuration file 210 in which the [REPEAT:n] tag is described. In this example, a [REPEAT:2] tag is described, and the MCU 30 recognizes that a count is executed twice with a default test setting (general viable bacteria [background: white]) for sample names “Rice ball” and “Fish-shaped bun”, and an average value of results thereof is obtained. In the count table 55, the MCU 30 arranges three count cells for each of “Rice ball” and “Fish-shaped bun”. The first count cell stores the first count result. The second count cell stores the second count result. The third count cell stores an average value of the first count result and the second count result.

In this manner, when the [REPEAT:n] tag is found during scanning of the configuration file 210, the MCU 30 arranges the n count rows for sample names described above a cell with the [REPEAT:n] tag in the cell group CG1 in the count table 55. Moreover, the MCU 30 also arranges a count row for storing the average value in the count table 55.

Note that, as illustrated in FIG. 27, the MCU 30 may arrange a number-of-times column for indicating names such as the first time, the second time, and the average value between a column for storing the sample name and a column for storing the count result.

(11) Configuration File without Tag

In the configuration file 210 described above, the MCU 30 creates the count table 55 by reading a tag such as [START], [END], [COUNT], or [COMMENT], and associates a predetermined test setting with the count cell 224. However, the tag may be omitted in the second embodiment.

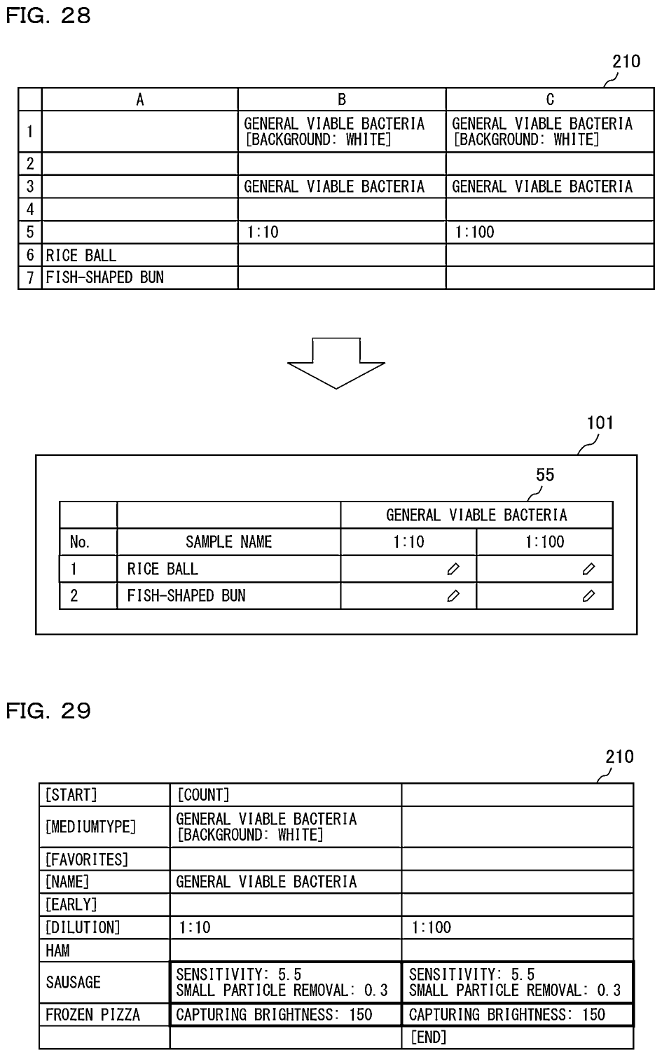

FIG. 28 illustrates a method of creating the count table 55 from the configuration file 210 including no tag. The storage device 35 stores a conversion rule in advance. For example, the conversion rule describes that a sample name is described in a column A, a name of a test setting is described in the first row, a name of a fungus is described in the third row, a dilution factor is described in the fifth row, and the like. Further, the conversion rule includes copying the sample name described in the column A to a column of a sample name in the count table 55, associating the test setting identified by the name in the first row with a count cell in the count table 55, copying a character string described in the third row to a cell of a fungus name in the count table 55, and copying a character string described in the fifth row to a cell of a dilution factor in the count table 55.

In this manner, the conversion rule has a rule indicating to which cell coordinates in the count table 55 a character string described in a cell of specific coordinates in the configuration file 210 is copied. Moreover, the conversion rule also has a rule indicating that a test setting identified by the character string described in the cell of the specific coordinates in the configuration file 210 is associated with the corresponding count cell 224 in the count table 55. The MCU 30 scans cells in the configuration file 210 according to the conversion rule, interprets the character string described in the cell of the specific coordinates according to the conversion rule, and reflects the character string in the count table 55.

As the conversion rule is stored in the storage device 35 in advance in this manner, the MCU 30 can convert the configuration file 210 into the count table 55 without using a tag. Alternatively, it can be said that the user needs to know the conversion rule and then input a character string according to the conversion rule to a predetermined cell in the configuration file 210.

### (12) Partial Customization of Test Setting

In FIG. 18, a test setting is designated by a combination of the [ON] tag and the [MEDIUMTYPE] tag, the [FAVORITES] tag, the [SAMPLEDB] tag, or a direct input of a name of the test setting. However, this is merely an example. The content of a test setting may be directly described in a test designation cell. For example, if a default test setting has J parameters, all of the J parameters may be described in the test designation cell. This means that the default test setting is substantially not used. Alternatively, K parameters among the J parameters may be described in the test designation cell (J>K>=1). In this case, the default test setting is applied to (J-K) parameters among the J parameters, and the test setting described in the test designation cell is applied to the K parameters. That is, it is possible to customize a part of the default test setting.

FIG. 29 illustrates an example of the configuration file 210 in which the default test setting can be partially or entirely customized. In this example, a setting value of the sensitivity and a threshold for small particle removal are directly described in test designation cells for a sample name “Sausage”. That is, only the sensitivity and the threshold for small particle removal in the test setting with the name of “General viable bacteria [background: white]” designated by the [MEDIUMTYPE] tag are customized to the values designated in the test designation cells. Capturing brightness is directly described in test designation cells for a sample name “Frozen pizza”. That is, only the capturing brightness in the test setting with the name of “General viable bacteria [background: white]” designated by the [MEDIUMTYPE] tag is customized to a value designated in the test designation cells.

In this manner, the default test setting can be partially or entirely customized by directly describing a test parameter in the test designation cell.

### (13) Flowchart

### (13-1) Main Flowchart

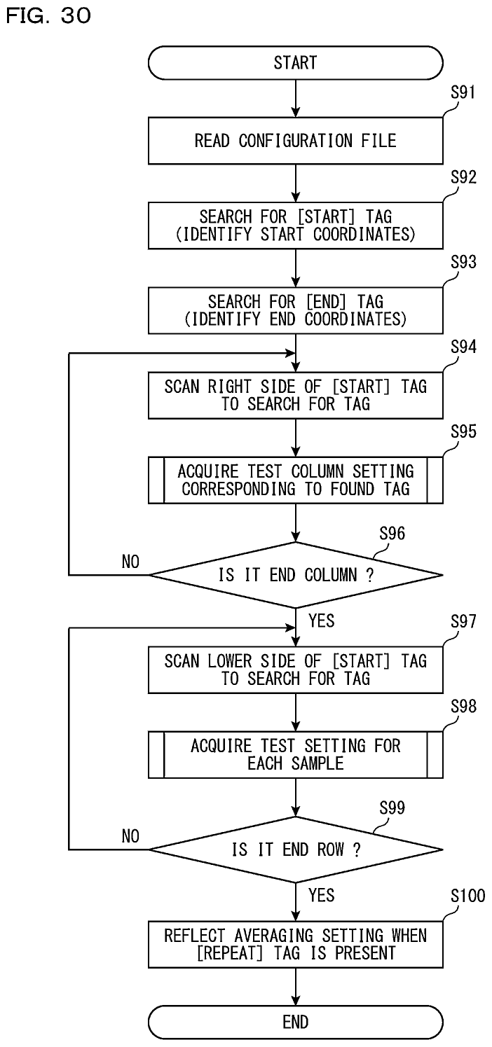

FIG. 30 illustrates processing of converting the configuration file 210, created by the user using the spreadsheet program 41, into the count table 55 by the application program 39.

In S91, the MCU 30 reads the configuration file 210 from the storage device 35. The configuration file 210 may be designated through a UI such as a file dialog. Note that the MCU 30 functions as a file reading section.

In S92, the MCU 30 searches the configuration file 210 for the [START] tag, and sets coordinates of a cell in which the [START] tag is described as start coordinates of the conversion processing. In this manner, the MCU 30 functions as a tag search section and a start coordinate setting section.

In S93, the MCU 30 searches the configuration file 210 for the [END] tag, and sets coordinates of a cell in which the [END] tag is described as end coordinates of the conversion processing. In this manner, the MCU 30 functions as the tag search section and an end coordinate setting section.

In S94, the MCU 30 starts scanning from the cell in which the [START] tag is described to the right side, and searches for a cell in which any tag is described. In this manner, the MCU 30 functions as a cell scanning section and the tag search section.

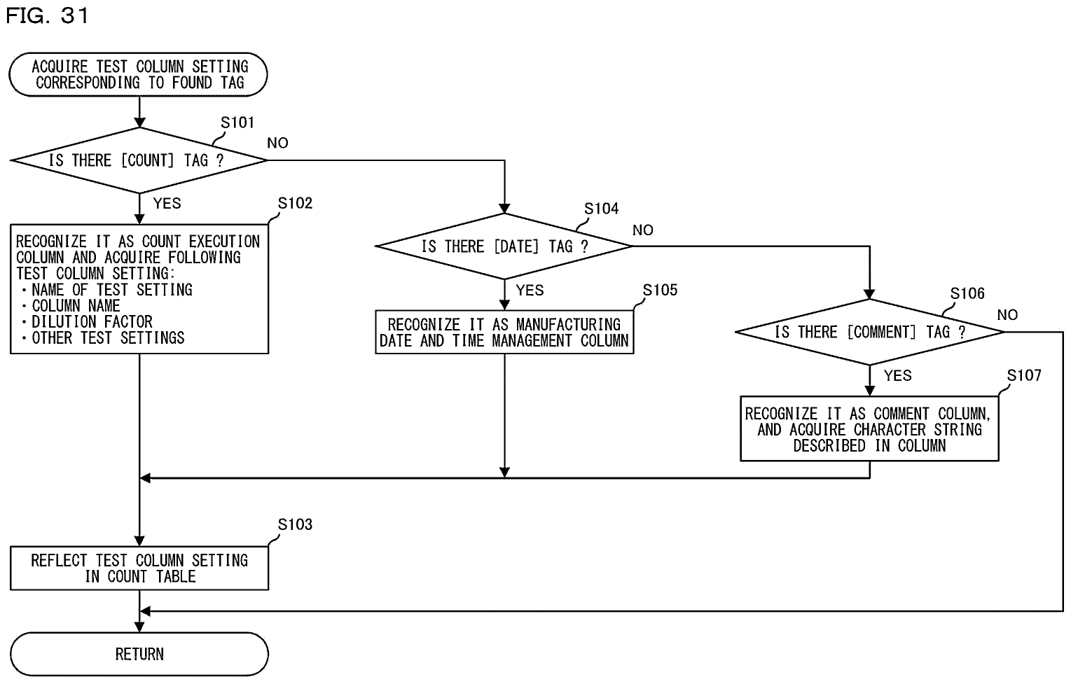

In S95, the MCU 30 acquires a test column setting corresponding to the found tag. Here, the test column setting is an information set (for example, a column name and the like) necessary for setting a corresponding column in the count table 55. According to FIG. 18, the [DATE] tag, the [COUNT] tag, and the [COUNT] tag are found. Details of S95 will be described later with reference to FIG. 31. In this manner, the MCU 30 functions as an acquisition section that acquires the test column setting.

In S96, the MCU 30 determines whether or not a scanning position reaches an end column (a column in which the [END] tag is described). When the scanning position is not the end column, the MCU 30 returns to S94, moves the scanning position to the right by one column, and searches for a tag. When the scanning position reaches the end column, the MCU 30 proceeds from S96 to S97. In this manner, the MCU 30 functions as a determination section.

In S97, the MCU 30 starts scanning to the lower side from the cell in which the [START] tag is described, and searches for a cell in which any tag or sample name is described.

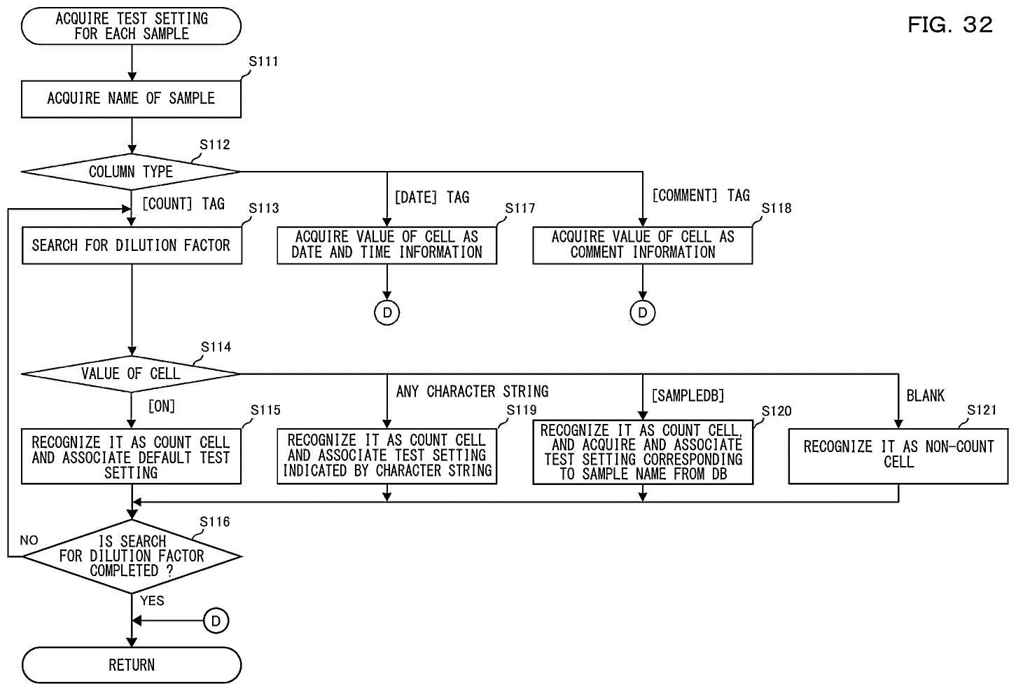

In S98, the MCU 30 acquires a test setting for each sample. Details of S98 will be described later with reference to FIG. 32. In this manner, the MCU 30 functions as a test setting acquisition section.

In S99, the MCU 30 determines whether or not the scanning position has reached an end row (a row in which the [END] tag is described). When the scanning position is not the end row, the MCU 30 returns to S97, moves the scanning position downward by one column, and searches for a tag. When the scanning position reaches the end column, the MCU 30 proceeds from S96 to S97.

In S100, when the MCU finds the [REPEAT] tag, the MCU 30 reflects an averaging setting in the count table 55. In this manner, the MCU 30 functions as a tag reflection section or an averaging setting reflection section.

### (13-2) Sub-Flowchart

FIG. 31 illustrates the details of S95.

In S101, the MCU 30 determines whether or not the [COUNT] tag has been found. When the [COUNT] tag is found, the MCU 30 proceeds from S101 to S102. Note that the MCU 30 proceeds to S102 also when a cell on the right side of a cell in which the [COUNT] tag is described is a blank cell.

In S102, the MCU 30 recognizes a column including the cell in which the [COUNT] tag is described as a count execution column, and acquires a test column setting (for example, a name of a default test setting, a column name (a name of bacterial species), a dilution factor, and other test settings) from the column.

In S103, the MCU 30 reflects the acquired test column setting in the count table 55.

In S101, when the found tag is not the [COUNT] tag, the MCU 30 proceeds from S101 to S104.

In S104, the MCU 30 determines whether or not the found tag is the [DATE] tag. When the [DATE] tag is found, the MCU 30 proceeds from S104 to S105.

In S105, the MCU 30 recognizes a column including a cell in which the [DATE] tag is described as a column for managing the manufacturing date and time. Thereafter, the MCU 30 proceeds from S105 to S103, and reflects the column for managing the manufacturing date and time in the count table 55. For example, the manufacturing date and time described in the column is copied to a corresponding column in the count table 55.

In S104, when the found tag is not the [DATE] tag, the MCU 30 proceeds from S104 to S106.

In S106, the MCU 30 determines whether or not the found tag is the [COMMENT] tag. When the [COMMENT] tag is found, the MCU 30 proceeds from S106 to S107.

In S107, the MCU 30 recognizes a column including a cell in which the [COMMENT] tag is described as a comment column, and acquires a character string (for example, a column name such as “Lot” or “Comment”) described in the column. Thereafter, the MCU 30 proceeds from S106 to S103, and reflects the comment column in the count table 55. For example, the character string (for example, the column name such as “Lot”) acquired from the comment column of the configuration file 210 is copied to a corresponding column in the count table 55.

### (13-3) Sub-Flowchart

FIG. 32 illustrates the details of S98.

In S111, the MCU 30 acquires a name of a sample from the configuration file 210. Note that a blank cell is skipped.

In S112, the MCU 30 determines a column type. When a tag indicating the column type is the [COUNT] tag, the MCU 30 proceeds from S112 to S113.

In S113, the MCU 30 searches for a dilution factor. For example, the dilution factor is acquired from a cell that is an intersection of the [COUNT] tag or the blank cell and the [DILUTION] tag.

In S114, the MCU 30 moves downward from the cell of the dilution factor, and branches the processing according to a value of the cell. When the value of the cell is [ON], the MCU 30 proceeds from S114 to S115.

In S115, the MCU 30 recognizes a cell in which the [ON] tag is described as a count cell (test designation cell), and associates the count cell with the default test setting designated by the [MEDIUMTYPE] tag. As a result, the default test setting is associated with the count cell in the count table 55 corresponding to the test designation cell in the configuration file 210. In this manner, the MCU 30 functions as an association section.

In S116, it is determined whether or not the search for the dilution factor has been completed. When there are a plurality of dilution factors for one sample name, the MCU 30 returns from S116 to S113.

When the value of the cell is any character string in S114, the MCU 30 proceeds from S114 to S119.

In S119, the MCU 30 recognizes the cell as the test designation cell, and associates the count cell 224 corresponding to the test designation cell with a test setting indicated by the character string described in the test designation cell. As a result, the test setting designated by a name in the test designation cell in the configuration file 210 is associated with the count cell 224 in the count table 55 corresponding to the test designation cell. Thereafter, the MCU 30 proceeds from S119 to S116.

When the value of the cell is [SAMPLEDB] in S114, the MCU 30 proceeds from S114 to S120.

In S120, the MCU 30 recognizes the cell as the test designation cell, acquires a test setting corresponding to the sample name from the sample DB 40 and associates the count cell 224 corresponding to the test designation cell with the acquired test setting. As a result, the test setting acquired from the sample DB 40 based on the sample name is associated with the count cell 224 in the count table 55 corresponding to the test designation cell. Thereafter, the MCU 30 proceeds from S120 to S116.

When the value of the cell is blank in S114, the MCU 30 proceeds from S114 to S121.

In S121, the MCU 30 recognizes the cell as a non-count cell. For example, the MCU 30 may gray out the non-count cell 226 in the count table 55 corresponding to the blank test designation cell. Thereafter, the MCU 30 proceeds from S121 to S116.

When it is determined in S112 that the tag indicating the column type is the [DATE] tag, the MCU 30 proceeds from S112 to S117.

In S117, the MCU 30 acquires the value of the cell as date and time information, and copies the date and time information to the corresponding count cell 224 in the count table 55. Thereafter, the MCU 30 proceeds from S117 to S116.

When it is determined in S112 that the tag indicating the column type is the [COMMENT] tag, the MCU 30 proceeds from S112 to S118.

In S118, the MCU 30 acquires the value of the cell as comment information, and copies the comment information to the corresponding count cell 224 in the count table 55. Thereafter, the MCU 30 proceeds from S118 to S116.

### SUMMARY

### [Viewpoint 1]

The head device 1a is an example of an acquisition section that acquires an image of colonies generated in a test individual. Note that the MCU 30 may function as the acquisition section by reading an image file of colonies designated by a user. The MCU 30 functions as an execution section that executes first software (for example, the application program 39), the execution section executing processing of counting the number of the colonies from the image of the colonies. The MCU 30 functions as a reading section that reads a user file (for example, the configuration file 210) which holds information in a matrix format and is created by second software (for example, the spreadsheet program 4) different from the first software in order to count the colonies generated in the test individual or to manage a count result of the colonies. Moreover, the MCU 30 functions as a creation section that creates the count table 55 to be used to count the colonies based on the user file. In this manner, as the configuration file 210 created by the spreadsheet program 41 different from the application program 39, which controls the colony counting device 1, is converted into the count table 55, burden of creating the count table 55 by the user is mitigated. As a result, the burden on the user regarding the counting of colonies is mitigated.

### [Viewpoint 2]

The storage device 35 functions as a storage section that stores a plurality of test settings (for example, General viable bacteria [background: white], General viable bacteria/setting 2, Escherichia coli group/dark culture medium) applied to the test individual. As illustrated in FIG. 20, the count table 55 includes a first cell indicating a type of the test individual, a second cell indicating a culture condition (for example, a dilution factor) applied to the test individual, and a third cell (for example, the count cell 224) that holds a count result of the colonies counted by applying the culture condition suggested by the second cell for the test individual suggested by the first cell. The MCU 30 functioning as the creation section associates the third cell with any one test setting of the plurality of test settings based on information described at a position corresponding to the third cell in the user file (for example, a test designation cell in the cell group CG3). As a result, the count cell 224 may be associated with the test setting using the configuration file 210.

### [Viewpoint 3]

As illustrated in FIG. 19, the user file may include a first cell group (for example, CG1 or the like) that holds type information indicating the type of the test individual, a second cell group (for example, CG2 or the like) that indicates a culture condition to be applied to the test individual, and a third cell group (for example, CG3 or the like) that holds setting information identified by a combination of the type of the test individual and the culture condition and associated with an imaging condition for acquiring an image to be applied to the test individual or a setting of image processing to be applied to the image. The MCU 30 functioning as the creation section may create the count table 55 by adopting the type information (for example, Sandwich set) held in the first cell group as a test individual name in the count table 55 and adopting a name (for example, 1:10, 1:100, or the like) of the culture condition held in the second cell group as a culture condition in the count table 55. In this manner, since a cell of the configuration file 210 corresponds to a cell of the count table 55, the user can create the configuration file 210 while being aware of the count table 55.

### [Viewpoint 4]

The setting of image processing may include a detection threshold (for example, binarization sensitivity) for detecting a colony as a count target from the image, or an exclusion threshold (for example, a threshold for small particle removal) of a particulate image excluded from the count target from the image.

### [Viewpoint 5]

The storage device 35 may function as a storage section that stores a plurality of pieces of setting information prepared in advance. Each of cells of the third cell group may be associated with any setting information of the plurality of pieces of setting information stored in the storage section based on an input value (for example, a name assigned to a test setting, the [SAMPLEDB] tag, a combination of the [ON] tag and the [MEDIUMTYPE] tag, and the like) of the cell. In this manner, the user can easily designate a test setting since there is a degree of freedom in a method of designating the test setting.

### [Viewpoint 6]

In the count table 55, the count cell 224 to which a count result for each combination of the test individual and the culture condition is input may be associated with setting information associated with a cell corresponding to the count cell in the third cell group of the user file. The MCU 30 clicks the icon 225 of the count cell 224 in the count table 55 to apply the test setting associated with the count cell 224 and cause the colony counting device 1 to execute a count. Here, the association between the count cell 224 and the test setting may be direct or indirect. In the former case, at the time of creating the count table 55, a test setting may be identified by a test designation cell of the cell group CG3 in the configuration file 210, and the identified test setting may be associated with the count cell 224. In the latter case, the count cell 224 may refer to the test designation cell of the cell group CG3 in the configuration file 210, and when the icon 225 is pressed, the MCU 30 may identify a test designation cell associated with the count cell 224 and further identify the test setting associated with the test designation cell.

### [Viewpoint 7]

The third cell group in the user file has a test designation cell corresponding to the count cell 224 in the count table 55. The test designation cell may hold a first character string (for example, [ON]). As illustrated in FIG. 19, a fourth cell group (for example, CG4) included in the user file may include a cell associated with any test setting among the plurality of test settings. For example, the cell is a cell (for example, the [MEDIUMTYPE] tag, or a cell in a row where the [FAVORITES] tag is present) corresponding to that the first character string (for example, [ON]) is described in the test designation cell. A character string (a name of the test setting (for example, General viable bacteria [background: white])) may be described in the cell. A test setting (for example, General viable bacteria/setting 1) associated with the character string described in this cell may be associated with the count cell 224. In this manner, a character string (for example, [ON]) indicating that a default test setting is designated may be described in the test designation cell.

### [Viewpoint 8]

The plurality of test settings may include a test setting uniquely created or selected by the user in accordance with the culture condition. As described above, the user may register some of the plurality of test settings as favorites. The test designation cell corresponding to the count cell 224 in the count table 55 in the third cell group in the user file may hold a second character string (for example, [ON]). In this case, a fifth cell group (for example, CG5) included in the user file is referred to. The fifth cell group also includes a cell associated with any one test setting of the plurality of test settings (for example, a cell in which a character string of Escherichia coli group/dark culture medium is described). The MCU 30 associates the count cell 224 with a test setting (for example, Escherichia coli group/dark culture medium) associated with the cell corresponding to the second character string. Note that, in the case of FIG. 19, a name of the default test setting is described in the cell group CG4 and names of the test settings registered as favorites are described in the cell group CG5. In the case of FIG. 19, the names of the test settings are described only in either the cell group CG4 or CG5. Therefore, when the [ON] tag is described in the cell group CG3, the names of the test settings described in the cell group CG4 or CG5 are identified. When the names of the test settings are described in both the cell group CG4 and the cell group CG5, the names of the test settings described in the cell group CG5 may be preferentially adopted.

### [Viewpoint 9]

As illustrated in FIG. 26, the test designation cell corresponding to the count cell 224 in the third cell group may hold a third character string (for example, [SAMPLEDB]). In this case, the MCU 30 associates the count cell 224 with a test setting associated with the test individual (for example, sample name) of the test designation cell corresponding to the count cell 224 among the plurality of test settings. In this manner, the count cell 224 may be associated with the test setting stored in the sample DB 40 or the like using the configuration file 210.

### [Viewpoint 10]

The test setting may be provided for each of types (for example, general viable bacteria, Escherichia coli group, and Staphylococcus aureus) of bacteria forming the colonies. The culture condition applied to the test individual may include a type of bacteria.

### [Viewpoint 11]

The culture condition applied to the test individual may include a combination of a type of bacteria and a dilution factor.

### [Viewpoint 12]

As illustrated in FIG. 19 and the like, in the user file, the third cell group (for example, CG3) may include a plurality of cells having different dilution factors for one type of test individual. Each of the plurality of cells may include a character string suggesting a test setting associated with a combination of the type of the test individual and the dilution factor. In FIG. 19, it is designated that a count is executed with three dilution factors (1:10, 1:100 and 1:1000) for Sandwich set. In this case, there are three test designation cells for three combinations, respectively. In FIG. 19, [ON] is described in each of the three test designation cells, but a name of a test setting such as General viable bacteria/setting 2 may be designated. Further, character strings described in the three test designation cells are not necessarily the same. That is, the three combinations may designate mutually different test settings.

### [Viewpoint 13]

The user file may include a culture condition in which a multiple counting mode (for example, the rapid mode) of executing a first count when a predetermined time shorter than a prescribed culture time elapses and executing a second count when the prescribed culture time elapses is set to be valid. In FIG. 19, the rapid mode is set to be valid by the [EARLY] tag and the [ON] tag.

As illustrated in FIG. 20, the creation section (the MCU 30) may create the count table 55 including a first count cell of the column 227 holding a result of the first count and a second count cell of the column 228 holding a result of the second count for the same test individual. In this manner, a plurality of columns according to the mode are automatically arranged in the count table 55 only by creating only one column in the configuration file 210.

### [Viewpoint 14]

The acquisition section may include an imaging section (for example, the main camera 11) that captures the test individual and generates a test image of the test individual. The MCU 30 may include a setting section that sets a first detection parameter for detecting colonies from a first test image of the test individual cultured for the predetermined time and a second detection parameter for detecting colonies from a second test image of the test individual cultured for the prescribed culture time. Here, a detection sensitivity of the first detection parameter is higher than a detection sensitivity of the second detection parameter. The MCU 30 may count the colonies by applying the first detection parameter to the first test image and input an intermediate result to the first count cell of the column 227, and counts the colonies by applying the second detection parameter to the second test image and inputs a final result to the second count cell of the column 228.

### [Viewpoint 15]

As illustrated in FIG. 24, the test individual may be accommodated in a test container (the Petri dish 15) divided into a plurality of accommodation regions by a separation wall. As illustrated in FIG. 25, the third cell group in the user file may include a cell for each of the accommodation regions, the cell being identified by a combination of the type of the test individual, the culture condition, and the accommodation region. The cell for each of the accommodation regions may include a character string (for example, “Upper left detection”) suggesting a test setting applied for each of the accommodation regions.

### [Viewpoint 16]

As described with reference to FIG. 25, in the third cell group of the user file, a test setting suggested by a character string (for example, “Upper left detection”) stored in a cell associated with a first accommodation region (for example, the upper left effective region 231) among the plurality of accommodation regions includes excluding a remaining region (for example, the effective regions 232 to 234) other than the first accommodation region among the plurality of accommodation regions from the count target. In the third cell group of the user file, a test setting suggested by a character string (for example, “Lower left detection”) stored in a cell associated with a second accommodation region (for example, the lower left effective region 234) among the plurality of accommodation regions includes excluding a remaining region (for example, the effective regions 231 to 233) other than the second accommodation region among the plurality of accommodation regions from the count target.

### [Viewpoint 17]

As described with reference to FIG. 22, the MCU 30 may function as a copy section that copies a count result to a test designation cell corresponding to the count cell 224 in the configuration file 210 when the count result is input to the count cell 224 included in the count table 55. Moreover, the MCU 30 may copy the count result to the business file 200.

### [Viewpoint 18]

As described with reference to FIG. 23, when a blank cell is found in the first cell group (for example, CG1), the creation section (the MCU 30) may create the count table 55 while ignoring a row including the blank cell.

### [Viewpoint 19]

As described with reference to FIG. 27, there is a case where the creation section (the MCU 30) finds a tag (for example, the [REPEAT:n] tag) that designates that a count is executed n times for the same test individual and a statistical value (for example, an average or a standard deviation) of n count results is obtained in the configuration file 210. In this case, the MCU 30 may arrange n cells for storing the n count results and a cell for storing the statistical value in the count table 55.

### [Viewpoint 20]

As described with reference to FIG. 28, the creation section (the MCU 30) may associate a test setting with the count cell 224 in which a count result is stored in the count table 55 based on a character string described in a cell located at a predetermined coordinate (for example, B1, C1, B3, C3, B5, C5, or A6 to A7) in the configuration file 210.

### [Viewpoint 21]

As described with reference to FIG. 29, at least one parameter (for example, Sensitivity: 5.5, Small particle removal: 0.3, Capturing brightness: 150, or the like) among a plurality of parameters constituting a test setting may be described in a cell included in the third cell group. In this case, the creation section (the MCU 30) may customize a part of the test setting associated with the cell to the parameter designated in the cell, and then associate the test setting with the count cell 224 corresponding to the cell in the count table 55.

### [Viewpoint 22]

The user file may be a CSV format file created using spreadsheet software (for example, the spreadsheet program 41) which is the second software or a file in a unique table format (for example, the xslx format) of the spreadsheet software. In particular, the xslx format has become widespread in the market and there are many users who are familiar with it. Therefore, the users can easily create the configuration file 210 used as the basis for the count table 55 using the familiar spreadsheet software.

### [Viewpoint 23]

As illustrated in FIG. 19, the configuration file 210 is an example of a configuration file that holds information in a plurality of cells of N rows×M columns. The configuration file 210 may include a first cell in which a type (for example, sample name) of a test individual is stored, a second cell in which a culture condition (for example, a dilution factor) of the test individual is stored, a third cell (for example, a cell in a row including the [NAME] tag) in which a bacterial species to be detected is stored, and a fourth cell which is identified by a combination of the type of the test individual, the culture condition of the test individual, and the bacterial species to be detected and suggests a test setting. For example, the fourth cell may be a test designation cell included in the cell group CG3, a cell in a row including the [MEDIUMTYPE] tag, or a cell in a row including the [FAVORITES] tag. The MCU 30 functions as a reading section that reads the configuration file 210. The storage device 35 may store a plurality of the test settings corresponding to at least one of the type of the test individual and the type of bacteria to be detected in order to identify an individual test setting corresponding to at least one of the type of the test individual and the type of bacteria to be detected. The MCU 30 functions as a count table generation section that generates the count table 55 including a plurality of candidate count cells (for example, the count cell 224) in which a count result is to be stored, the count result being counted by a test based on the type of the test individual, the culture condition of the test individual, the bacterial species to be detected, and the test setting suggested by the fourth cell, defined by the combination of the type of the test individual, the culture condition of the test individual, and the bacterial species to be detected, among the plurality of test settings included in the configuration file 210 read by the reading section. Here, each of the plurality of candidate count cells is identified by the combination of the type of the test individual, the culture condition of the test individual, and the bacterial species to be detected. The display control section 36 functions as a display control section that displays the count table 55 generated by the count table generation section (MCU 30) on a display section (for example, the display device 37). The pointing device 33 functions as a cell identifying section that identifies one cell to which a count result is input from among the plurality of candidate count cells included in the count table 55 displayed by the display control section 36. The MCU 30 functions as a test execution section that acquires a test image that is an image of the test individual based on a test setting and counts colonies included in the test image. Moreover, the MCU 30 functions as a count table editing section that allocates a count result of the colonies counted by the test execution section to the one cell identified by the cell identifying section.

### [Viewpoint 24]

The MCU 30 functions as a reading section that reads a configuration file having a type of a test individual, a culture condition of the test individual, and a bacterial species to be detected as items of either a row or a column and having a first test setting corresponding to an array defined by the items. The storage device 35 stores a second test setting corresponding to at least one of the type of the test individual and the bacterial species to be detected in order to identify an individual test setting corresponding to each of types of a plurality of the test individuals and each of a plurality of the bacterial species to be detected. The MCU 30 may function as a count table generation section that generates the count table 55 including a plurality of candidate count cells in which a count result is to be stored, the count result being counted by a test based on the type of the test individual, the culture condition of the test individual, the bacterial species to be detected, and the first test setting (for example, Information for designating a test setting stored in cell group CG3) corresponding to the array defined by a combination of the type of the test individual, the culture condition of the test individual, and the bacterial species to be detected, which are included in the configuration file read by the reading section, and the second test setting (for example, an actual test setting) corresponding to the first test setting and stored in the storage section.

The invention is not limited to the above embodiment, and various modifications and changes can be made within a scope of a gist of the invention.

What is claimed is:

1. A colony counting device comprising:
an acquisition section configured to acquire an image of colonies generated in a test individual; and
an execution section configured to execute first software, the execution section being configured to execute count processing of a number of the colonies from the image of the colonies,
wherein the execution section includes:
a reading section configured to read a user file that holds information in a matrix format and is created by second software different from the first software in order to count the colonies generated in the test individual or to manage a count result of the colonies; and
a creation section configured to create a count table to be used to count the colonies based on the user file.

2. The colony counting device according to claim 1, further comprising a storage unit configured to store a plurality of test settings applied to the test individual,
wherein the count table includes a first cell indicating a type of the test individual, a second cell indicating a culture condition applied to the test individual, and a third cell holding a count result of colonies counted by applying the culture condition suggested by the second cell to the test individual suggested by the first cell, and
the creation section associates the third cell with any one test setting of the plurality of test settings based on information described at a position corresponding to the third cell in the user file.

3. The colony counting device according to claim 1, wherein
the user file includes:
a first cell group holding type information indicating a type of the test individual;
a second cell group indicating a culture condition applied to the test individual; and
a third cell group holding a test setting identified by a combination of the type of the test individual and the culture condition, the test setting being associated with an imaging condition for acquiring the image applied to the test individual or a setting of image processing applied to the image, and
the creation section creates the count table by adopting the type information held in the first cell group as a test individual name in the count table and adopting a name of the culture condition held in the second cell group as a culture condition in the count table.

4. The colony counting device according to claim 3, wherein the setting of image processing includes a detection threshold for detecting a colony as a count target from the image or an exclusion threshold of a particulate image excluded from the count target from the image.

5. The colony counting device according to claim 3, further comprising a storage section configured to store a plurality of test settings prepared in advance,
wherein each of cells of the third cell group is associated with any test setting of the plurality of test settings stored in the storage section based on an input value of each of the cells.

6. The colony counting device according to claim 5, wherein in the count table, a count cell to which a count result for each combination of the test individual and the culture condition is input is associated with the test setting associated with a cell, which corresponds to the count cell, in the third cell group of the user file.

7. The colony counting device according to claim 6, wherein, when the cell, which corresponds to the count cell in the count table, in the third cell group of the user file holds a first character string, the count cell is associated with a test setting associated with a cell, which corresponds to the first character string, in a fourth cell group including a cell associated with any test setting among the plurality of test settings, the fourth cell group being included in the user file.

8. The colony counting device according to claim 6, wherein
the plurality of test settings include a test setting created or selected by a user in accordance with the culture condition, and
when the cell, which corresponds to the count cell in the count table, in the third cell group of the user file holds a second character string, the count cell is associated with a test setting associated with a cell, which corresponds to the second character string, in a fifth cell group including a cell associated with any test setting among the plurality of test settings, the fifth cell group being included in the user file.

9. The colony counting device according to claim 6, wherein, when the cell, which corresponds to the count cell, in the third cell group holds a third character string, the count cell is associated with a test setting associated with the test individual of the cell corresponding to the count cell among the plurality of test settings.

10. The colony counting device according to claim 1, wherein, when the user file includes a culture condition in which a multiple counting mode of executing a first count when a predetermined time shorter than a prescribed culture time elapses and executing a second count when the prescribed culture time elapses is set to be valid, the creation section creates a count table that includes a first count cell holding a result of the first count and a second count cell holding a result of the second count for the same test individual.

11. The colony counting device according to claim 10, wherein
the acquisition section includes an imaging section configured to capture the test individual and generates a test image of the test individual,
the execution section includes a setting section configured to set a first detection parameter for detecting the colonies from a first test image of the test individual cultured for the predetermined time and a second detection parameter for detecting the colonies from a second test image of the test individual cultured for the prescribed culture time,
a detection sensitivity of the first detection parameter is higher than a detection sensitivity of the second detection parameter, and
the execution section further counts the colonies by applying the first detection parameter to the first test image and inputs an intermediate result to the first count cell, and counts the colonies by applying the second detection parameter to the second test image and inputs a final result to the second count cell.

12. The colony counting device according to claim 3, wherein
the test individual is accommodated in a test container divided into a plurality of accommodation regions by a separation wall, and
the third cell group of the user file includes a cell for each of the accommodation regions, the cell being identified by a combination of the type of the test individual, the culture condition, and the accommodation region, and the cell for each of the accommodation regions includes a character string suggesting a test setting applied for each of the accommodation regions.

13. The colony counting device according to claim 12, wherein
in the third cell group of the user file, a test setting suggested by a character string stored in a cell associated with a first accommodation region among the plurality of accommodation regions includes excluding, from a count target, a remaining region other than the first accommodation region among the plurality of accommodation regions, and
in the third cell group of the user file, a test setting suggested by a character string stored in a cell associated with a second accommodation region among the plurality of accommodation regions includes excluding, from the count target, a remaining region other than the second accommodation region among the plurality of accommodation regions.

14. The colony counting device according to claim 3, further comprising a copy section configured to copy a count result to a cell, which corresponds to a count cell, in the user file when the count result is input to the count cell included in the count table.

15. The colony counting device according to claim 1, wherein the creation section arranges n cells for storing n count results and a cell for storing a statistical value in the count table when a tag designating that a count is executed n times for the same test individual and the statistical value of the n count results is obtained is found in the user file.

16. The colony counting device according to claim 1, wherein the creation section associates a test setting with a count cell in which a count result is stored in the count table based on a character string described in a cell located at a predetermined coordinate in the user file.

17. The colony counting device according to claim 3, wherein when at least one parameter of a plurality of parameters constituting a test setting is described in a cell included in the third cell group, the creation section customizes a part of the test setting associated with the cell to the at least one parameter, and then associates the test setting with a count cell, which corresponds to the cell, in the count table.

18. The colony counting device according to claim 1, wherein the user file is a CSV format file created using spreadsheet software that is the second software or a file in a unique table format of the spreadsheet software.

19. A colony counting device comprising:
a reading section configured to read a configuration file holding information in a plurality of cells of N rows×M columns, the configuration file including a first cell in which a type of a test individual is stored, a second cell in which a culture condition of the test individual is stored, a third cell in which a bacterial species to be detected is stored, and a fourth cell which is identified by a combination of the type of the test individual, the culture condition of the test individual, and the bacterial species to be detected and suggests a test setting;
a storage section configured to store a plurality of test settings corresponding to at least one of the type of the test individual and the bacterial species to be detected in order to identify an individual test setting corresponding to the at least one of the type of the test individual and the bacterial species to be detected;
a count table generation section configured to generate a count table including a plurality of candidate count cells in which a count result is to be stored, the count result being counted by a test based on the type of the test individual, the culture condition of the test individual, the bacterial species to be detected, and the test setting suggested by the fourth cell, defined by the combination of the type of the test individual, the culture condition of the test individual, and the bacterial species to be detected, among the plurality of test settings included in the configuration file read by the reading section, each of the plurality of candidate count cells being identified by the combination of the type of the test individual, the culture condition of the test individual, and the bacterial species to be detected;
a display control section configured to display the count table generated by the count table generation section on a display section;
a cell identifying section configured to identify one cell to which the count result is input from among the plurality of candidate count cells included in the count table displayed by the display control section;
a test execution section configured to acquire a test image that is an image of the test individual based on the test setting and count colonies included in the test image; and
a count table editing section configured to allocate the count result of the colonies counted by the test execution section to the one cell identified by the cell identifying section.

20. A colony counting device comprising:
a reading section configured to read a configuration file having a type of a test individual, a culture condition of the test individual, and a bacterial species to be detected as items of either a row or a column and having a first test setting corresponding to an array defined by the items;
a storage section configured to store a second test setting corresponding to at least one of the type of the test individual and the bacterial species to be detected in order to identify an individual test setting corresponding to each of types of a plurality of the test individuals or each of a plurality of the bacterial species to be detected;
a count table generation section configured to generate a count table including a plurality of candidate count cells in which a count result is to be stored, the count result being counted by a test based on the type of the test individual, the culture condition of the test individual, the bacterial species to be detected, and the first test setting corresponding to the array defined by a combination of the type of the test individual, the culture condition of the test individual, and the bacterial species to be detected, which are included in the configuration file read by the reading section, and the second test setting corresponding to the first test setting and stored in the storage section, each of the plurality of candidate count cells being identified by the combination of the type of the test individual, the culture condition of the test individual, and the bacterial species to be detected;
a display control section configured to display the count table generated by the count table generation section on a display section;
a cell identifying section configured to identify one cell to which the count result is input from among the plurality of candidate count cells included in the count table displayed by the display control section;
a test execution section configured to acquire a test image that is an image of the test individual based on the second test setting and count colonies included in the test image; and
a count table editing section configured to allocate the count result of the colonies counted by the test execution section to the cell identified by the cell identifying section.
